(19) United States
(12) Patent Application Publication
(10) Pub. No.: US 2026/0107187 A1
(43) Pub. Date: Apr. 16, 2026

(54) COMPRESSION MECHANISM FOR THERAPY DATA

(71) Applicant: Resmed Sensor Technologies Limited, Sandyford, Dublin (IE)

(72) Inventors: Shahil MAHARAJ, Dublin (IE); Romain Geoffrey Pierre DOUMENC, Dublin (IE)

(73) Assignee: Resmed Sensor Technologies Limited, Sandyford, Dublin (IE)

(21) Appl. No.: 19/117,282

(22) PCT Filed: Oct. 2, 2023
(86) PCT No.: PCT/EP2023/077274

§ 371 Date: Mar. 31, 2025

Related U.S. Application Data
(60) Provisional application No. 63/412,704, filed on Oct. 3, 2022.

(51) Int. Cl. H04W 28/06; A61M 16/00

(52) U.S. Cl. CPC H04W 28/06; A61M 16/024; A61M 2205/3553; A61M 2205/3584; A61M 2205/502; A61M 2205/6072

(57) ABSTRACT

A data gathering system (100) has at least one processor configured to perform a compression scheme for wireless transmission. The compression scheme may include amplitude reduction, where a difference between every two consecutive values of a plurality of values is computed and stored in an array. Additionally, the compression scheme may include bit packing, where outlier values are output in a first bit length, and remaining values are output in a second bit length, where the second bit length is determined so as to minimize a total memory cost for all values. Further, the system may store data recorded at different points in time in an array of data chunks, and wirelessly transmit the array of data chunks simultaneously.

### 1 CROSS REFERENCE TO RELATED APPLICATIONS

This application claims the benefit of U.S. Provisional Patent Application No. 63/412,704, filed Oct. 3, 2022, the entire content of which is incorporated herein by reference.

### 1.1 FIELD OF THE TECHNOLOGY

The present technology generally relates to systems for transmission of data, such as physiological data, between devices such as by using a compression scheme for efficient transmission of data, such as therapy and/or usage data recorded by a respiratory therapy system over a low-bandwidth connection, without loss of precision of the underlying data.

### 1.2 DESCRIPTION OF RELATED ART

Home-based respiratory therapy devices allow patients to receive respiratory treatments at the comfort of the patients' home. To check for compliance or to monitor conditions of the patients, clinicians need to regularly review therapy data collected by the respiratory therapy devices.

Some existing respiratory therapy devices rely on a modem, such as a Global System for Mobile Communications (GSM) chip, to transfer therapy data to a remote server that is accessible by the clinician. The modem may be a built-in modem integrated into the therapy devices. Alternatively, the modem may reside in a wireless plug-in dongle that is releasably attached to the therapy devices. Certain plug-in dongles do not have wireless transmission capabilities. Instead of wirelessly transmitting the therapy data, such dongles are first manually inserted into the therapy devices to obtain the therapy data, and then manually inserted into a wireless device, such as a desktop computer, to upload the therapy data to the remote server. Other existing respiratory devices encode and display the therapy data in the form of barcode such as quick response (QR) code, scannable by a wireless device such as a smart phone, which then transmits the scanned data to the remote server. However, due to bandwidth limitations of such transfer mechanisms, such as in the nature of the modem's connection with a network, due to encoding limitations, or due to capacity constraints of the plug-in dongles, the existing transfer mechanisms are not always able to transfer high resolution data. For example, a respiratory therapy device may have collected an extensive quantity of pressure and flow data samples from one or more sensors over one or more multiple treatment sessions that span across days or weeks, where each treatment session may last for hours such as when a treatment session corresponds with a night of sleep. Due to various limitations described above, the respiratory therapy device is limited to transfer low resolution data, such as a summary or subset of the samples, as opposed to sending all or a substantial portion of the samples.

Such devices may benefit from a compression scheme that reduces the effective payload size of high resolution data for effective transmission over a low-bandwidth connection, without losing precision of the underlying data.

### 2 BRIEF SUMMARY OF THE TECHNOLOGY

The present technology presents a lossless data compression scheme for efficient transmission of data over a low-bandwidth connection.

Some implementations of the present technology may include a system. The system may include a data transfer device for transferring data from the system. The system may include at least one processor. The at least one processor may be configured to compress a plurality of values for the data transfer device. The at least one processor may be configured to compute a difference between every two consecutive values of the plurality of values. The at least one processor may be configured to output an array to represent compression of the plurality of values. The output array may include a first value of the plurality of values, and the computed difference.

In some implementations, the data transfer device may include any of a wireless transceiver, a removable memory medium, and a display coupled with the at least one processor so as to be configured to display the data in a format readable by a wireless device. The format readable by the wireless device may include one or more of a barcode, text and a symbol. The barcode may include a quick response (QR) code. The at least one processor is configured to compute the difference between every two consecutive values by using an XOR function to obtain a differentiating bit sequence. Each value may include 12 bits, for example. The plurality of values may include the first value and a second value. The output array may include, in a first position, the first value, and wherein the output array may include, in a second position, the computed difference between the first value and the second value.

In some implementations, each element in the output array may correspond to a value of the plurality of values at a corresponding position. A first element in a first position of the output array may store the first value of the plurality of values. A succeeding element therefrom in a succeeding position of the output array may store the computed difference between a corresponding value of the plurality of values and its preceding value. The system may include a respiratory pressure medical device. The plurality of values may include any one or more of: interaction data, therapy data, and device information. The device information may include any one or more of, or at least one of: one or more device settings, one or more device factory settings, a device identifier, and operational data.

In some implementations, the at least one processor may be configured to reduce a total memory use for storing the output array, by identifying a first bit length to represent at least one outlier value of the output array. The at least one processor may be configured to reduce a total memory use for storing the output array, by determining a second bit length to represent at least one remaining value of the output array, the second bit length being less than the first bit length. The at least one processor may be configured to reduce a total memory use for storing the output array, by outputting each outlier value in the first bit length. The at least one processor may be configured to reduce a total memory use for storing the output array, by outputting each remaining value in the second bit length.

In some implementations, the output values may be recorded at different points in time, wherein the at least one processor may be configured to group the output values into an array of data chunks, the array of data chunks may include a first data chunk and a plurality of succeeding data chunks, the first data chunk may include overall information describing the array of data chunks, each succeeding data chunk being configured to store the output values recorded at one of the different points in time. The output values may be recorded at different points in time, wherein the at least one processor may be configured to instruct the data transfer device to wirelessly transmit the array of data chunks simultaneously. The array of data chunks may include a header. The header may include at least one of the following, or one or both of: the first bit length and the second bit length.

Some implementations of the present technology may include a system. The system may include a data transfer device for transferring data from the system. The system may include at least one processor. The at least one processor may be configured to compress a plurality of values for the data transfer device by reducing a total memory use for storing the plurality of values. The at least one processor may be configured to identify a first bit length to represent at least one outlier value of the plurality of values. The at least one processor may be configured to determine a second bit length to represent at least one remaining value of the plurality of values, the second bit length being less than the first bit length. The at least one processor may be configured to output each outlier value in the first bit length. The at least one processor may be configured to output each remaining value in the second bit length.

In some implementations of the system, the data transfer device may include any of a wireless transceiver, a removable memory medium, and a display coupled with the at least one processor so as to be configured to display the data in a format readable by a wireless device. Each outlier value may be a value of the plurality of values that exceeds the second bit length but fits in the first bit length. Each remaining value may be a value of the plurality of values that fits in the second bit length. The total memory use (or a total memory cost) for storing) may be a sum of a first product and a second product, wherein the first product is a product of the first bit length and a total number of the at least one outlier value, and wherein the second product is a product of the second bit length and a total number of the at least one remaining value. The at least one processor may be configured to pad or remove at least one data bit to or from the at least one outlier value of the plurality of values to satisfy the first bit length. The at least one processor may be configured to pad or remove at least one data bit to or from the at least one remaining value of the plurality of values to satisfy the second bit length. The first bit length may be based on a bit position of a most significant bit in a maximum value of the plurality of values.

Some implementations of the present technology may include a system. The system may include a transceiver configured to transmit data wirelessly. The system may include at least one processor. The at least one processor may be configured to group data, recorded at different points in time, into an array of data chunks. The array of data chunks may include a first data chunk and a plurality of succeeding data chunks. The first data chunk may include overall information describing the array of data chunks, each succeeding data chunk may be configured to store the data recorded at one of the different points in time. The at least one processor may be configured to instruct the transceiver to wirelessly transmit the array of data chunks simultaneously.

In some implementations, each succeeding data chunk may store at least one of, or one or more: time at which each data is recorded, and a bit length of each value contained in the data. At least one of the data chunks may include an array of data. A first data chunk of the array of data chunks may include a header. The header may include at least one of, or one or more of, the following: a device identifier, an array size, a size of each data chunk, a key for encryption and/or signature and a checksum for data integrity.

Some implementations of the present technology may include a method. The method may include compressing, by one or more processors of a data gathering system, a plurality of values for data transfer. The compressing may include computing a difference between every two consecutive values of the plurality of values. The compressing may include outputting an array to represent compression of the plurality of values. The output array may include a first value of the plurality of values, and the computed difference. The method may include controlling a transfer of the output array with a data transfer device configured to transfer data from the data gathering system.

In some implementations, the method may reduce a total memory use (or a total memory cost) for storing the output array by identifying a first bit length to represent at least one outlier value of the output array. The method may reduce a total memory use for storing the output array by determining a second bit length to represent at least one remaining value of the output array, the second bit length being less than the first bit length. The method may reduce a total memory use for storing the output array by outputting each outlier value in the first bit length. The method may reduce a total memory use for storing the output array by outputting each remaining value in the second bit length.

In some implementations, the output values may be recorded at different points in time, and wherein the method may further include grouping the output values into an array of data chunks. The array of data chunks may include a first data chunk and a plurality of succeeding data chunks. The first data chunk may include overall information describing the array of data chunks. Succeeding data chunks, such as each, may be configured to store the output values recorded at one of the different points in time. The method may include instructing the data transfer device to wirelessly transmit the array of data chunks simultaneously. The method may further include generating a header for the array of data chunks, and wherein the header may include at least one of the following: the first bit length and the second bit length.

Some implementations of the present technology may include a method. The method may include compressing, by one or more processors of a data gathering system, a plurality of values for data transfer to reduce a total memory use for storing the plurality of values. The compressing may include identifying a first bit length to represent at least one outlier value of the plurality of values. The method may include determining a second bit length to represent at least one remaining value of the plurality of values, the second bit length being less than the first bit length. The compressing may include outputting each outlier value in the first bit length. The compressing may include outputting each remaining value in the second bit length. The method may include controlling transfer of each output outlier value and each output remaining value with a data transfer device configured to transfer data from the data gathering system.

In some implementations, each outlier value may be a value of the plurality of values that exceeds the second bit length but fits in the first bit length. Each remaining value may be a value of the plurality of values that fits in the second bit length. In some implementations, the total memory cost may be a sum of a first product and a second product, wherein the first product may be a product of the first bit length and a total number of the at least one outlier value, and wherein the second product may be a product of the second bit length and a total number of the at least one remaining value. The method may further include padding or removing at least one data bit to or from the at least one outlier value of the plurality of values to satisfy the first bit length. The method may further include padding or removing at least one data bit to or from the at least one remaining value of the plurality of values to satisfy the second bit length. The first bit length may be based on a bit position of a most significant non-zero bit in a maximum value of the plurality of values.

Some implementations of the present technology may include a method. The method may include grouping, by one or more processors of a data gathering system, data recorded at different points in time into an array of data chunks. The array of data chunks may include a first data chunk and a plurality of succeeding data chunks. The first data chunk may include overall information describing the array of data chunks. Succeeding data chunks, such as each, may be configured to store the data recorded at one of the different points in time. The method may include wirelessly transmitting, by a transceiver, the array of data chunks simultaneously.

In some implementations, the method may include storing at least one of, or one or both of, the following in the array of data chunks: time at which each data is recorded, and a bit length of each value contained in the data. Each data chunk may include an array of data. A first data chunk of the array of data chunks may include a header. The header may include at least one of the following: a size of the array, a size of each data chunk, a key for encryption and/or signature, and a checksum for data integrity.

Some implementations of the present technology may include a processor-readable medium, having stored thereon processor-executable instructions which, when executed by the one or more processors of the data gathering system, cause the one or more processors to perform any one or more of the aspects of any of the methods described herein.

Some implementations of the present technology may include a system. The system may include a wireless transceiver for transmitting data from the system. The system may include
at least one processor configured to compress a plurality of values for transmission by the wireless transceiver. The at least one processor may be configured to compute a difference between every two consecutive values of the plurality of values. The at least one processor may be configured to output an array to represent compression of the plurality of values. The output array may include a first value of the plurality of values, and the computed difference.

In some implementations, the at least one processor may be configured to reduce a total memory use for storing the output array by identifying a first bit length to represent at least one outlier value of the output array. The at least one processor may be configured to reduce a total memory use for storing the output array by determining a second bit length to represent at least one remaining value of the output array. The second bit length may be less than the first bit length. The at least one processor may be configured to reduce a total memory use for storing the output array by outputting each outlier value in the first bit length. The at least one processor may be configured to reduce a total memory use for storing the output array, by outputting each remaining value in the second bit length.

In some implementations, the output values may be recorded at different points in time. In such a case, the at least one processor may be configured to group the output values into an array of data chunks. The array of data chunks may include a first data chunk and a plurality of succeeding data chunks. The first data chunk may include overall information describing the array of data chunks, succeeding data chunks, such as each, may be configured to store the output values recorded at one of the different points in time. The at least one processor may be configured to instruct the data transfer device to wirelessly transmit the array of data chunks simultaneously.

Some implementations of the present technology may include a system. The system may include, may include a removable memory medium for receiving data from the system. The system may include at least one processor. The system may include at least one processor may be configured to compress a plurality of values for storage in the removable memory medium. The at least one processor may be configured to compute a difference between every two consecutive values of the plurality of values. The system may include at least one processor may be configured to output an array to represent compression of the plurality of values, the output array may include a first value of the plurality of values, and the computed difference.

In some implementations, the removable memory medium may be configured to wirelessly transmit the compressed values. The at least one processor may be configured to reduce a total memory use for storing the output array, by identifying a first bit length to represent at least one outlier value of the output array. The at least one processor may be configured to reduce a total memory use for storing the output array, by determining a second bit length to represent at least one remaining value of the output array. The second bit length may be less than the first bit length. The at least one processor may be configured to reduce a total memory use for storing the output array, by outputting each outlier value in the first bit length. The at least one processor may be configured to reduce a total memory use for storing the output array, by outputting each remaining value in the second bit length.

In some implementations, the output values may be recorded at different points in time. In such a case, the at least one processor may be configured to group the output values into an array of data chunks. The array of data chunks may include a first data chunk and a plurality of succeeding data chunks. The first data chunk may include overall information describing the array of data chunks. Succeeding data chunks, such as each, may be configured to store the output values recorded at one of the different points in time. The at least one processor may be configured to instruct the data transfer device to wirelessly transmit the array of data chunks simultaneously.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 2.1 BRIEF DESCRIPTION OF DRAWINGS

The present technology is illustrated by way of example, and not by way of limitation, in the figures of the accompanying drawings, in which same reference numerals refer to same elements in construction and in operation, and like reference numerals refer to similar elements in construction and in operation, including:

FIG. 1A shows an example environment in which an implementation of a data gathering system sends data to a target system;

FIG. 1B shows an example environment in which an implementation of the data gathering system sends data to the target system;

FIG. 1C shows an example environment in which an implementation of the data gathering system sends data to the target system;

FIG. 1D shows an example environment illustrating an implementation of the data gathering system;

FIG. 2 shows a block diagram of the data gathering system, such as a respiratory pressure therapy (RPT) device;

FIG. 3 shows example input and output arrays of an amplitude reduction phase of a compression scheme;

FIG. 4 shows example input and output arrays of a bit packing phase of the compression scheme;

FIG. 5 shows an example output array of a bundling phase of the compression scheme;

FIG. 6 shows an example compression process performed on multiple time series data;

FIG. 7A illustrates a flow diagram of an example process for compressing data in the amplitude reduction phase;

FIG. 7B illustrates a flow diagram of an example process for compressing data in the bit packing phase;

FIG. 7C illustrates a flow diagram of an example process for bundling data recorded at different points in time;

FIG. 8A shows an example system in accordance with the present technology. A patient 1000 wearing a patient interface 3000 receives a supply of pressurised air from an RPT device 4000. Air from the RPT device 4000 is humidified in a humidifier 5000, and passes along an air circuit 4170 to the patient 1000. A bed partner 1100 is also shown.

FIG. 8B shows an RPT device 4000 in use on a patient 1000 with a nasal mask 3000.

FIG. 8C shows an RPT device 4000 in use on a patient 1000 with a full-face mask 3000.

FIG. 9 shows an example non-invasive patient interface 3000 in the form of a nasal mask.

FIG. 10A shows an RPT device 4000 in accordance with one form of the present technology.

FIG. 10B shows a schematic diagram of the pneumatic circuit of an RPT device 4000 in accordance with one form of the present technology. The directions of upstream and downstream are indicated.

FIG. 10C shows a schematic diagram of the electrical components of an RPT device 4000 in accordance with one aspect of the present technology.

FIG. 10D shows a schematic diagram of the algorithms 4300 implemented in an RPT device 4000 in accordance with an aspect of the present technology. In FIG. 10D, arrows with solid lines indicate an actual flow of information, for example via an electronic signal.

FIG. 10E is a flow chart illustrating a method 4500 carried out by the therapy engine module 4320 of FIG. 10D in accordance with one aspect of the present technology.

FIG. 11 shows a humidifier 5000.

### 3 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 1. Overview

One aspect of the present technology relates to a compression process methodology that adapts data recorded by a data gathering system, such a physiological data determined with one or more sensors, to a reduced payload size, without loss of data pression, for efficient transmission over to a low-bandwidth connection.

### 1.1 Data Gathering System

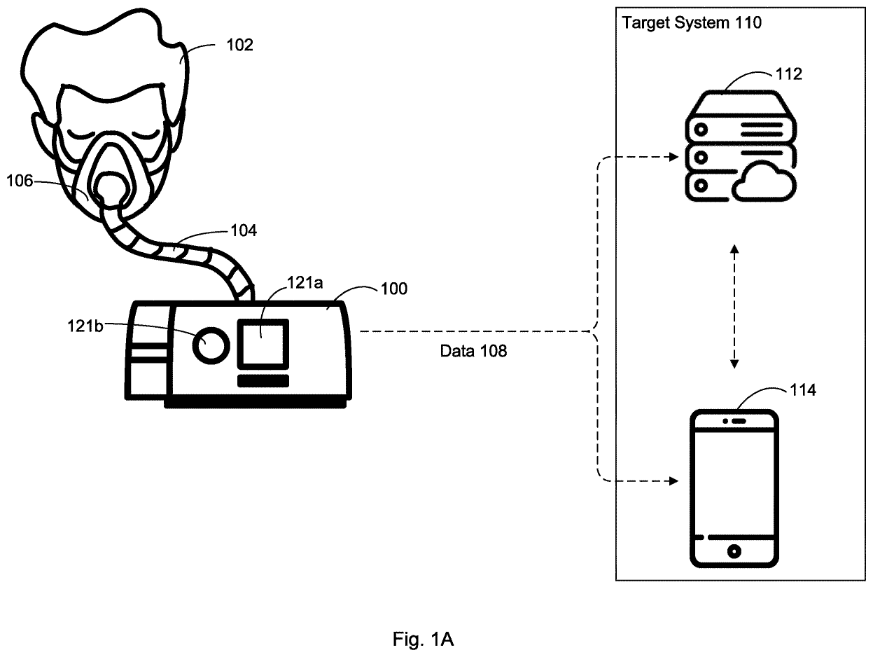

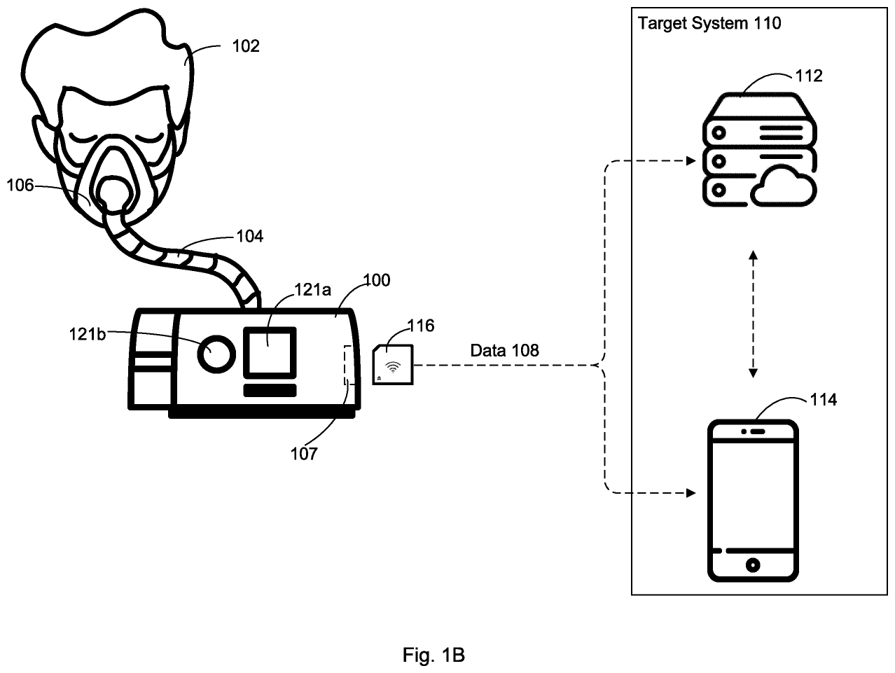

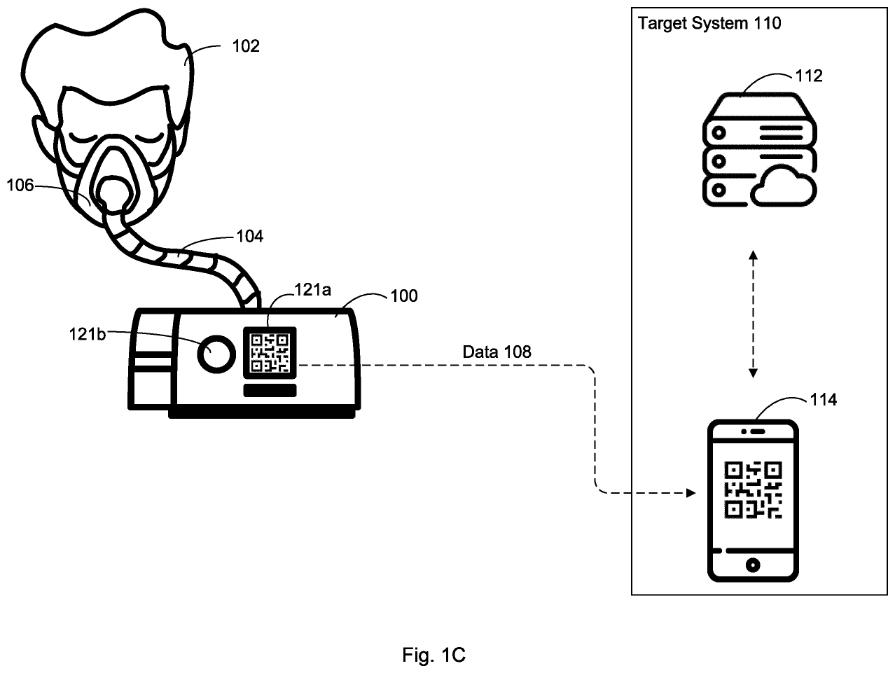

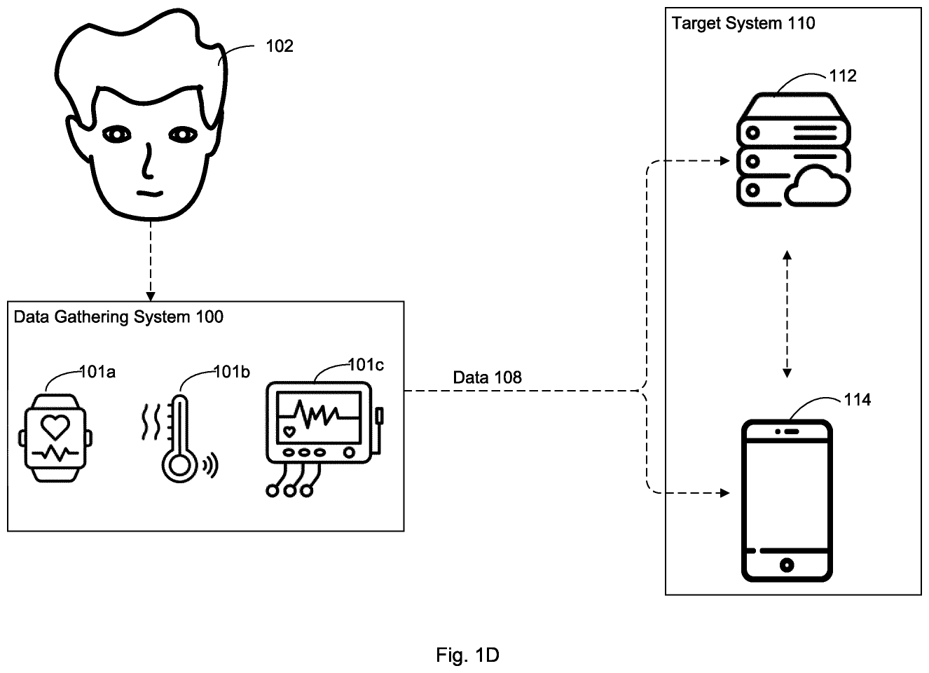

FIGS. 1A-D illustrate various examples of a data gathering system 100 that is configured to receive, detect, compress or transfer data 108. Such data 108 may include time varying numeric signals, such as physiological data.

In some implementations as illustrated in FIG. 1A, the data gathering system 100 may provide a flow of breathable gas to a user 102 via an air circuit 104. For example, the data gathering system 100 may include, but not limited to, a respiratory pressure medical device, such as a respiratory therapy (RT) device, a respiratory pressure therapy (RPT) device, and/or a high flow therapy device (HFT) described herein, that provides respiratory therapy to the user 102. An interface 106, such as a mask, may be used to interface the data gathering system 100 to the user 102. Depending upon the therapy to be applied, the interface 106 may form a seal, e.g., with a face region of the user 102, to facilitate the delivery of gas at a pressure at sufficient variance with ambient pressure to effect therapy. In such implementations, the data 108 transferred by the data gathering system 100 may include, but not limited to, therapy data associated with the therapy provided to the user 102, which may include physiological data.

The data gathering system 100 may send data 108 to a target system 110 via a wired or wireless connection. In one example, the data gathering system 100 may include a built-in wireless transceiver, which may be regarded as a data transfer device, configured to transmit the data 108 wirelessly. The target system 110 may include one or more of the following a remote server 112 and/or a wireless device 114. FIG. 1B illustrates another implementation of the data gathering system 100, which is similar to the prior implementations, except that the implementations may include a memory card slot 107. The memory card slot 107 may be configured to removably receive a removable memory medium 116, which may also be referred to as a removable memory card, and may also be referred to as a data transfer device. The data gathering system 100 may store data 108 onto the removable memory medium 116 which, in turn, may provide the data 108 to the target system 110. In some such implementations, the removable memory medium 116 may have a built-in wireless transceiver configured to wirelessly transfer the data 108 to the target system 110. In some implementations, the removable memory medium 116 may transfer the data 108 to the wireless device 114 when manually inserted into the wireless device 114. The wireless device 114 may in turn wirelessly transmit the data 108 to the remote server 112.

FIG. 1C illustrates another implementation of the data gathering system 100, which is similar to the prior implementations of FIG. 1A, except that such implementations may display the data 108 through its display 121a in a format readable by the wireless device 114. Such format may include but not limited to, barcode such as QR code, text and symbol, or other scannable graphic on a display the encodes data, among other possibilities. The wireless device 114 may read and/or scan the display 121a, transmit the scanned data and/or any data decoded from the scanned data to the remote server 112. The display 121a may be regarded as a data transfer device.

In another implementation as illustrated in FIG. 1D, the data gathering system 100 may be any system that is configured to receive, detect, compress or transfer physiological data associated with the user 102. Such physiological data may include, but not limited to, heart rate, step count, blood glucose, blood pressure, respiration rate, body temperature, blood volume, sound pressure, photoplethysmography, electroencephalogram, electrocardiogram, blood oxygen saturation, and skin conductance, among other possibilities. For example, the data gathering system 100 may include one or more of the following: a smart watch 101a, a temperature sensor 101b, an electrocardiogram (ECG/EKG) device 101c, a health tracker, and a smart phone, among other possibilities. The data gathering system 100 may transfer data 108, such as the physiological data, to the target system 110 through a built-in wireless transceiver as described in the first embodiment, a removable memory medium as described in the second embodiment, or a display as described in the third embodiment. Each of the built-in wireless transceiver, the removable memory medium and the display as described above may regarded as a data transfer device.

### 1.2 Remote Server

The remote server 112 may be a remotely located computing system of one or more servers that receives data 108 provided by one or more data gathering systems 100. The remote server 112 may be implemented to monitor conditions or treatment progress of one or more users based on data 108 provided by one or more data gathering systems 100. The remote server 112 may be a cloud-based server system. The remote server 112 may be accessible to a clinician(s).

Each user may have a user account at the remote server 112. Each user account may store historical data 108 obtained from the data gathering system 100, so that the remote server 112 can track treatment progress of each individual user.

### 1.3 Wireless Device

The wireless device 114 may be a computing system accessible by the user 102. The wireless device 114 may collect, manage and/or monitor data 108 provided by the user's data gathering system 100. Examples of the wireless device 114 may include mobile phone, tablet, netbook, desktop computer, laptop computer, and wearable computing device such as a smartwatch, among other possibilities. The wireless device 114 may include all of the components normally used in connection with a computing device such as a user interface subsystem.

In one example, when the wireless device 114 is connected with the data gathering system 100, the wireless device 114 may have two-way communication with the data gathering system 100. The wireless device 114 may transmit any user input, including any device setting of the data gathering system 100, to the data gathering system 100. The data gathering system 100 may receive the user's input via the wireless device 114, and adjust any device setting according to the user input. The data gathering system 100 may send data 108 to the wireless device 114. The wireless device 114 may display, in its graphical user interface, data 108 received from the data gathering system 100 to the user 102. Alternatively, the wireless device 114 may process data 108 received from the data gathering system, and display any processed data to the user 102.

Additionally, or as an alternative, the wireless device 114 may forward the data 108 received from the data gathering system 100 to the remote server 112. In another example, after the wireless device 114 processes the data 108 received from the data gathering system, the wireless device 114 may send the processed data to the remote server 112.

### 2 Data Gathering System

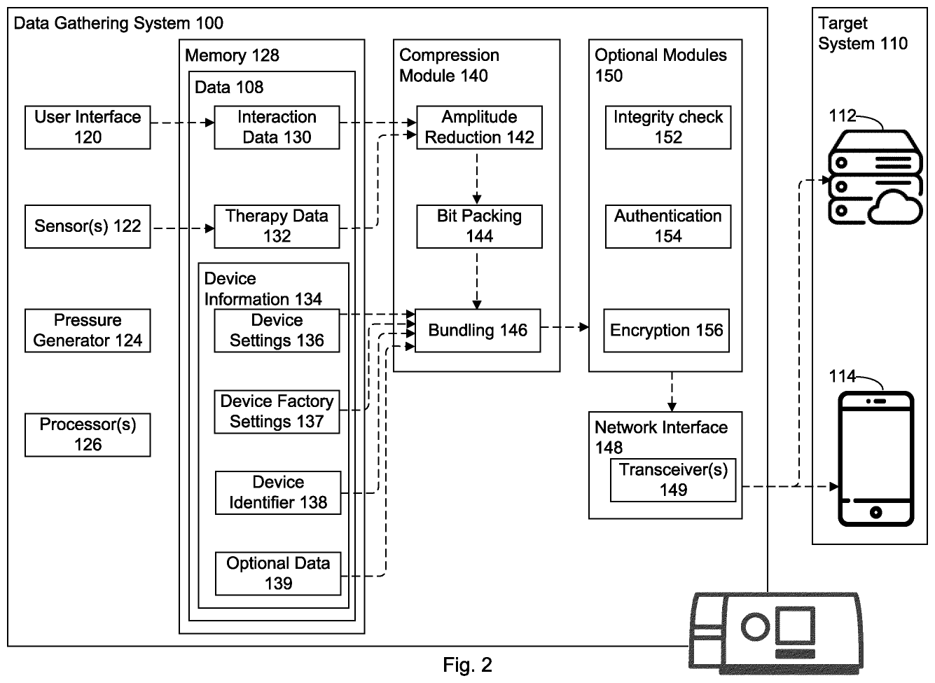

Referring to FIG. 2, the aforementioned implementations of the data gathering system 100 may include any one or more of the following: a user interface 120, one or more sensors 122, a pressure generator 124, one or more processors 126, memory 128, one or more compression modules 140, a network interface 148, and one or more optional modules 150, among others.

### 2.1 User Interface

The user interface 120 may include one or more of the following: a display 121a for presenting a graphical user interface and one or more menu selectors 121b, as shown in FIG. 1. The selector(s) 121b may, for example, take the form of a knob or a button, which may be manipulated by the user 102 to operate the data gathering system 100. The user 102 may manipulate the selector(s) 121b to navigate and/or select menus displayed in the display 121a. In one embodiment, the display 121a may have a touch screen.

### 2.2 Sensor

The sensor(s) 122 may be configured to generate sensor measurements. In some implementations, the sensor measurement(s) may include determined physiological data, that may be sensed directly or derived from sensed signals, such as respiratory measurements of the user 102. The respiratory measurements may include, but not limited to, any one or more of a flow rate of a pressurized flow of breathable gas delivered to the user 102, a pressure of breathable gas as generated by the pressure generator 124 and/or as measured or estimated at the user's mouth, a patient respiratory flow rate, and/or how many respiratory events the user has experienced, such as the number of respiratory events per hour. The sensor measurement(s) may also include the user's heart rate, which may be measured directly or inferred from other data, such as cardiogenic oscillations in the flow rate data.

The sensor measurement(s) may include other physiological data, including, but not limited to, heart rate, step count, blood glucose, blood pressure, respiration rate, body temperature, blood volume, sound pressure, photoplethysmography, electroencephalogram, electrocardiogram, blood oxygen saturation, and skin conductance, among other possibilities.

### 2.3 Pressure Generator

The pressure generator 124 may be configured to generate a pressurized flow of breathable gas for delivery to the airway of the user 102. In one example, the pressure generator 124 may generate the pressurized flow according to a target pressure waveform. The target pressure waveform may represent varying pressure of the flow of breathable gas that the pressure generator aims to produce.

### 2.4 Processor

The processor(s) 126 may be any conventional processors, such as commercially available GPUs, CPUs, TPUs, etc. Alternatively, each processor may be a dedicated device such as an ASIC or other hardware-based processor. References to a processor may be understood to include references to a collection of processors that may or may not operate in parallel.

### 2.5 Memory

The memory 128 may include databases that store information accessible by the processor(s) 126. The memory 128 may store data 108 and instructions for performing one or more of the following: data compression, encryption, authentication, and integrity check, among others.

The memory 128 may be of any type capable of storing information accessible by the processor(s) 126, including a computing device-readable medium. The memory may be a non-transitory medium such as a hard-drive, memory card, optical disk, solid-state, or other memory etc. The memory may include different combinations of the foregoing, whereby different portions of instructions and data are stored on different types of media. The instructions may be any set of instructions to be executed directly (such as machine code) or indirectly (such as scripts) by the processor(s). For example, the instructions may be stored as computing device code on the computing device-readable medium. In that regard, the terms “instructions”, “modules” and “programs” may be used interchangeably herein. The instructions may be stored in object code format for direct processing by the processor, or in any other computing device language including scripts or collections of independent source code modules that are interpreted on demand or compiled in advance.

Although FIG. 2 functionally illustrates the processor(s) 126 and the memory 128 as being within the same block, such devices may actually include multiple processor or memories that may or may not be stored within the same physical housing. Similarly, the memory 128 may be a hard drive or other storage media located in a housing different from that of the processor(s) 126, for instance in a cloud computing system. The processor(s) 126 may respectively access the memory 128 via a network.

In one example, the memory 128 may include a memory card that is removably inserted into a memory card slot of the data gathering system 100. The memory card may slide in and out of the memory card slot to engage or disengage respectively with the data gathering system 100. For instance, the memory card may be dimensioned and configured as any of a removable flash memory card and a universal serial bus (USB) flash drive, among other possibilities. The memory card may communicate with the data gathering system 100 via a data communication interface of the data gathering system 100. The memory card may include a non-volatile memory for storing data 108.

### 2.5.1 Data

Data 108 may include but not limited to, interaction data 130, therapy data 132 related to one or more treatment sessions of the user 102, and device information 134, among other possibilities. Data 108 may be encoded in the form of barcode such as QR code, and/or in text format, among other possibilities and displayed on a display of the data gathering system 100 for transfer to another device such as the wireless device 114.

Data 108 may include multiple timeseries signals. In one example, data 108 may be encoded using a standard format for time series. For instance, data 108 may be encoded using bytes, such as little or big endian, to represent integers. Sequences of integers may be stored as a stream of bytes, where each byte may encode an integer.

Data 108 may be stored in a fixed size. For example, data 108 may be stored as fixed precision numbers.

In one example, data 108 may be stored on a physical flash drive, and may be retrieved by standard operating system calls which map the data into the memory 128.

### 2.5.1.1 Interaction Data

Interaction data 130 may include, but not limited to, the user's input entered through the user interface 120 such as the display 121a and/or the menu selector(s) 121b.

### 2.5.1.2 Therapy Data

Therapy data 132 may include, but not limited to, one or more sensor measurements as provided by the sensor(s) 122, information determined based on the sensor measurement(s), parameters of the user 102 as collected by the data gathering system 100, usage data of the data gathering system 100, and/or events of the data gathering system 100.

Usage data may include how much time the data gathering system 100 has been used such as usage hours, how much time the interface 106 has been worn by the user 102 during one or more treatment sessions, the number of times that the interface 106 is on and off the user 102, efficiency of mask seal, and any mask leak information, among others. In one example, usage data may be derived from one or more sensor measurements provided by the sensor(s) 122.

### 2.5.1.3 Device Information

Device information 134 may include one or more of the following: one or more device settings 136, one or more device factory settings 137, a device identifier 138, and optional data 139, among other possibilities.

### 2.5.1.3.1 Device Setting

The device settings 136 may control the pressure generated by the pressure generator 124. The user 102 may manually adjust one or more device settings through the user interface 120 and/or the data gathering system 100 may automatically adjust settings in response to detected conditions. The device settings 136 may include one or more of the following parameters of the data gathering system 100 used in the user's treatment session(s): therapy mode, maximum pressure, minimum pressure, and expiratory pressure relief (EPR) parameters, among other possibilities.

The EPR parameters may include, but not limited to, an inspiratory pressure trigger threshold, an inspiratory pressure shape, a peak inspiratory pressure peak, an expiratory pressure trigger threshold, an expiratory pressure shape, and a peak expiratory pressure. The inspiratory pressure trigger threshold may be a parameter that indicates when the pressure generator 124 starts to generate an inspiratory pressure to assist the user's inspiration during the user's inspiration cycle. The inspiratory pressure shape may refer to a parameter that determines the shape of the inspiratory pressure curve from the start of the inspiratory pressure to the peak inspiratory pressure. The peak inspiratory pressure may include a first parameter that controls when to generate the peak inspiratory pressure, and a second parameter that controls an amplitude of the peak inspiratory pressure, which may represent the amount of pressure supplied by the pressure generator 124 at the peak time. The expiratory pressure trigger threshold may be a parameter that controls when the pressure generator 124 starts pressure reduction to assist the user's expiration during the user's expiration cycle. The peak expiratory pressure may include a first parameter representing the amplitude of the minimum expiratory pressure generated by the pressure generator 124 during the user's expiration, or how far the pressure falls during the user's expiration, and a second parameter indicating when to generate the peak expiratory pressure. The expiratory pressure shape may refer to the shape of the expiratory pressure curve from the end of the inspiratory pressure to the peak expiratory pressure.

### 2.5.1.3.2 Device Factory Setting

Device factory settings 137 may include one or more of the following parameters of the data gathering system 100 preprogramed by the manufacturer: mode, maximum pressure, minimum pressure, and EPR parameters, among other possibilities.

### 2.5.1.3.3 Device Identifier

The device identifier 138 may uniquely identify the data gathering system 100. An example of the device identifier may be a serial number of the data gathering system 100. In some implementations, the device identifier 138 may be omitted or may be a unique identifier other than the serial number such as a patient identifier. In one example, the device identifier 138 may be acquired by capturing an image of a label on the data gathering system 100. Alternatively, the device identifier 138 may be entered by the user 102 into the data gathering system 100, through keyboard input or audio input. In one example, the device identifier 138 may be sent to the target system 110, such as the remote server 112, to complete registration of the data gathering system 100.

2.5.1.3.4 Optional data

Optional data 139 may include, but not limited to, an encryption key to perform encryption, network authentication information, error correction and/or detection coding information.

The data gathering system 100 may encrypt any data by using the encryption key before transmitting the data to the target system 110 for secured transmission.

The data gathering system 100 may use the network authentication information to access a network, and communicate with the target system 110 via the network.

The data gathering system 100 may send error correction and/or detection coding information to the target system 110 to verify integrity of any transmitted data.

### 2.5.1.4 Additional Data

In addition to the various types of data described above, data 108 may also include physiological data to track general wellbeing of the user 102. In this regard, such physiological data may be obtained with one or more sensors 122, which may be outside of or independent from any treatment session of the user 102. In some instances, such data may also be obtained during a treatment session. Such physiological data may include any one or more of, but not limited to, heart rate, step count, blood glucose, blood pressure, respiration rate, body temperature, blood volume, sound pressure, photoplethysmography, electroencephalogram, electrocardiogram, blood oxygen saturation, and/or skin conductance, among other possibilities.

### 2.6 Network Interface

The network interface 148 may be configured to establish a wired or wireless communication with the target system 110. The network interface may have one or more transceivers 149, such as a Bluetooth transceiver, a cellular transceiver and a Wi-Fi communication transceiver. The transceiver(s) 149 may include an antenna adapted to wirelessly transmit and receive data packets. The transceiver(s) 149 may be regarded as a data transfer device. In one example, the network interface 148 may be configured to detect and join a wired or wireless network so as to form a wired or wireless communication with the target system 110. In one example, the network interface 138 may form a low-bandwidth communication with the target system 110.

### 2.7 Compression Module

The compression module 140, executable by the processor(s) 126, may perform compression on the data 108. Compression may take place prior to encoding the data 108 in any particular format such as barcode. Compression may include one or more of the following phases: amplitude reduction 142, bit packing 144 and bundling 146, each of which is discussed in detail below.

### 2.7.1 Amplitude Reduction

The data 108 may include an array of values. In the amplitude reduction phase 142, the processor(s) 126 may compress the array of values by implementing a delta coding technique, which determines a difference between two consecutive values. The processor(s) 126 may output the difference between each pair of two consecutive values to an output array.

To determine the difference between two consecutive values, the processor(s) 126 may determine an arithmetic difference between the two values by subtracting each value from its previous value.

Alternatively, the processor(s) 126 may perform an XOR function on two consecutive values. The XOR function may result in a differentiating bit sequence between the two values. For example, a first value may be 1056, represented as 010000100000 in the binary form. A second value may be 1032, represented as 010000001000 in the binary form. An XOR operation between 1056 and 1032 may result in 40, represented as 000000101000 in the binary form.

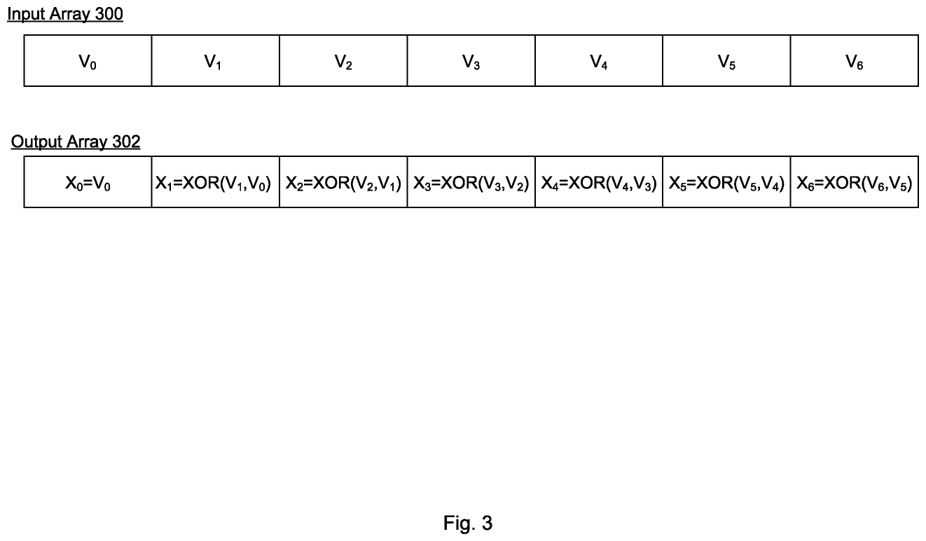

Referring to FIG. 3, the processor(s) 126 may compress an input array 300 of values V₀, V₁, . . . . Vₙ by generating an output array 302 that stores compressed values X₀, X₁, . . . . Xₙ of the input array 300. The output array 302 may also be referred to as an amplitude reduced array 302. The input array 300 may represent all or a partial set of the data 108 stored in the memory 128. All values of the input array 300 may have the same bit length. For instance, each value of the input array 300 may comprise 16 bits. In one example, the input array 300 may include a large set of integers, such as a block of 2048 bytes.

The processor(s) 126 may generate the output array 302 according to equations (1) and (2) provided below.

$${X}_{0} = {V}_{0} \qquad ( 1 )$$

$${X}_{n + 1} = XOR ( {V}_{n + 1} , {V}_{n} ) \qquad ( 2 )$$

Each element in the output array 302 may correspond to an element in the input array 300 at the same position. The first element X₀ in the first position of the output array 302 may store the first value V₀ from the input array 300.

Each succeeding element in a succeeding position of the output array 302 may store a compressed value of a corresponding element in the input array 300. The compressed value may be the difference between the corresponding input value and its preceding input value. For example, the n+1^{th} element Xₙ₊₁ in the output array 302 may store the difference between Vₙ₊₁ and Vₙ resulted from the XOR operation.

As shown in FIG. 3, the second element X₁ in the output array 302 may store the difference between V₁ and V₀. The third element X₂ may store the difference between V₂ and V₁. The fourth element X₃ may store the difference between V₃ and V₂. The fifth element X₄ may store the difference between V₄ and V₃. The sixth element X₅ may store the difference between V₅ and V₄. The seventh element X₆ may store the difference between V₆ and V₅.

In one embodiment, the outcome of the amplitude reduction phase may not yield improvements in space per se, since the values in the output array 302 may have the same size as values in the input array 300. For example, if each value of the input array 300 is encoded as a 16-bit integer, each value of the output array 302 may still be a 16-bit integer, even if most of the significant bits are unused. To reduce space, the processor(s) may perform further compression, as discussed below.

### 2.7.2 Bit Packing Phase

The bit packing phase 144 may be configured to reduce the total number of bytes and/or bits used to represent values.

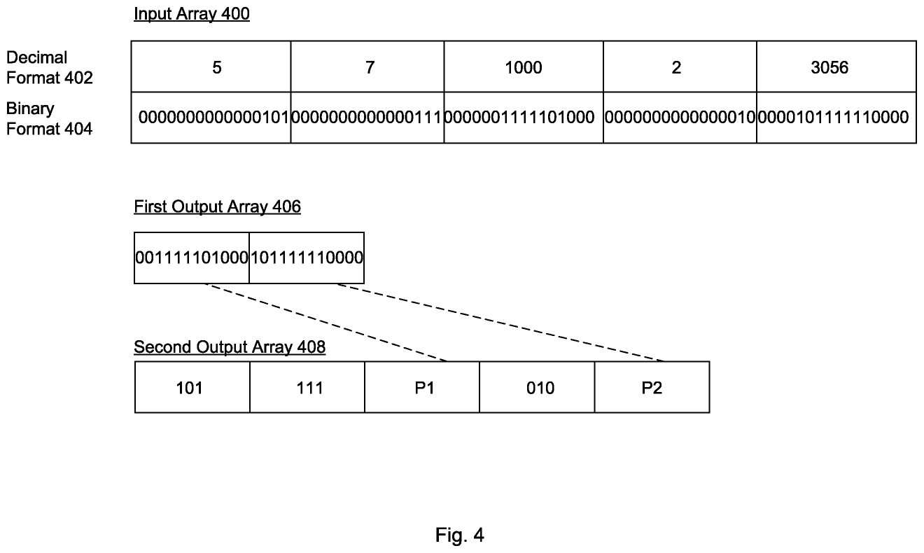

Referring to FIG. 4, in the bit packing phase 144, the processor(s) 126 may receive an input array 400 of values. The input array 400 of values may be the amplitude-reduced array 302 resulted from the amplitude reduction phase. For illustration purposes only, FIG. 4 shows decimal format 402 and binary format 404 of each value in the input array 400.

To compress the input array 400, the processor(s) 12 may generate two output arrays 406 and 408. The first output array 406 may be used to may store large values in the input array 400. Each value of the first output array 406 may be stored by using a first, relatively large bit length.

To determine the first bit length, the processor(s) may find the maximum value in the input array 400, and deduce the minimum number of bits needed to represent the maximum value. In one example, the minimum number of bits to represent the maximum value may be based on a bit position of a most significant non-zero bit in the maximum value. For instance, with reference to FIG. 4, the maximum value in the input array 400 is 3056, which is expressed as 0000101111110000 in the binary form. Its most significant non-zero bit is the 12th bit from left. As such, the minimum number of bits to represent 3056 is 12 bits. As such, the first bit length in this example is 12. As such, the first output array 402 is a 12-bit output array, where each element in the output array 406 comprises 12 bits. The first output array 406 may be referred to as an exception frame. The values stored in the first output array 406 may be referred to as outlier values.

Not all values in the input array 400 need to be expressed using 12 bits. Values such as 5 and 7 in the input array 400 may be sufficiently expressed using 3 bits. Using 12 bits to represent such values would dramatically increase the overall memory cost, resulting in a huge waste of memory space.

As a result, the processor(s) 124 may generate a second output array 408 for storing remaining values, which may be referred to as standard values. Each value of the second output array 408 may be stored by using a second, relatively small bit length. To determine the second bit length, the processor(s) 126 may search for the best number of bits that can minimize the total memory cost C_{b} for all values, provided in equation (3)

$${C}_{b} = ( n - {n}_{b} ) \times B + {n}_{b} \times b \qquad ( 3 )$$

In equation (3), B may represent the first bit length for expressing outlier values, b may represent the second bit length for expressing standard values, n may represent the total number of values in the input array 400, n_{b} may represent the number of values that fit into b bits, and C_{b} may represent the total memory cost for storing all values including outlier values and standard values. In one embodiment, the second bit length b may range from 0 to a B−1. When the second bit length b is zero, the processor(s) 124 may output a single output array, where each element in the output array is expressed using the first bit length.

As shown in equation (3), the total memory cost C_{b} may be a sum of a first product and a second product. The first product (n−n_{b})×B may be a product of the first bit length B and a total number of outlier values as represented by n−n_{b}. The second product n_{b}×b may be a product of the second bit length b and a total number n_{b} of all standard values.

In one embodiment, after determining the first bit length B based on the minimum number of bits needed to represent the maximum value in the input array, the processor(s) 126 may use a standard binary search algorithm to find the second bit length b that minimize the total memory cost C_{b}, in logarithmic time.

After determining the second bit length, the processor(s) 126 may identify each outlier value from the input array 400 by searching for any value that exceeds the second bit length but fits in the first bit length. For example, the processor(s) 126 may determine the bit location of the most significant non-zero bit for each value in the input array 400. For any value that has a most significant non-zero bit location greater than the second bit length, that value may be deemed as an outlier value. The processor(s) 126 may output each outlier value into the first output array 406 in the first bit length. In this process, the processor(s) 126 may convert each outlier value by expressing it using the first bit length. During the conversion process, to satisfy the first bit length, the processor(s) 126 may pad one or more data bits to outlier value(s) or remove one or more empty prefix data bits, such as most significant zero bits, from the outlier value(s).

The processor(s) 126 may identify each standard value from the input array 400 by searching for any value that fits in the second bit length. For any value in the input array 400 that has a most significant non-zero bit location less than or equivalent to the second bit length, that value may be deemed as a standard value. The processor(s) 126 may output each standard value into the second output array 408 in the second bit length. In this process, the processor(s) 126 may convert each standard value by expressing it using the second bit length. During the conversion process, to satisfy the second bit length, the processor(s) 126 may pad one or more data bits to the standard value(s), or remove one or more empty prefix data bits, such as most significant zero bits, from the standard value(s). By way of example, the number 7 in a 5-bit binary format is 00111. To express the number 7 in a 3-bit length, the first two prefix bits 00 may be removed from the 5-bit binary format 00111.

In the case of FIG. 4, the first bit length is 12 as discussed above, and the total number of values in the input array is 5. The processor(s) 126 may find that when the second bit length is 3, the total memory cost C_{b} reaches its minimum value. The processor(s) 126 may identify all outlier values, namely, 1000 and 3056, that exceeds the second bit length, and output them into the first output array 406. The processor(s) 126 may identify all standard values namely, 5, 7 and 2, that fit into the second bit length, and output them into the second output array 408.

As shown in FIG. 4, each element in the second output array 408 may correspond to an element in the input array 400 at the same position. For example, values 5, 7 and 2 remain at the same position in the second output array 408 as in the input array 400. Values 1000 and 3056 although are not stored in the second output array 408, their respective addresses P1 and P2 in the first output array 406 are stored in the second output array 408. Their positions in the second output array 408 are consistent with their positions in the input array 400. For instance, value 1000 is at the third position in the input array 400, and its address in the first output array 406 is stored at the third position in the second output array 408. Similarly, value 3056 is at the fifth position in the input array 400, and its address in the first output array 406 is stored at the fifth position in the second output array 408.

When the input array 400 has a plurality of values within close proximity, e.g., a block of 2048 values that are all below 8. The minimum total memory cost occurs when all these values are expressed by using the same bit length. For example, all values may be sufficiently expressed by using 4 bits. The processor(s) 124 may find that the first bit length to be 4, and the second bit length to be 0. As a result, the processor(s) may output a single output array, and each value in the single output array may be expressed using the first bit length.

The bit packing phase as described above may be particularly helpful for compressing constant data, or data with small variations such as the user's heart rate.

### 2.7.3 Bundling

The data gathering system 100 may send data 108 to the target system 110 as a bundle. Each bundle may be a single output array that can be transmitted in a single transmission to the target system 110 at a single point in time.

In one embodiment, a bundle may contain only one type of data 108. For example, the bundle may include only device settings 136, or only a subset of therapy data 132. In another embodiment, a bundle may contain a combination of different types of data 108, such as a combination of device settings 136 and therapy data 132. The choice of a specific combination may be driven by the mechanism of the connection between the data gathering system 100 and the target system 110.

In one embodiment, a bundle may include data 108 recorded at multiple points in time. For instance, the bundle may include therapy data of multiple treatment sessions, which may span across multiple days, such as 180 days.

In one embodiment, each array output from the bit packing phase 144 may belong to a single time series. Each time series may include one or more of the following recorded during the same treatment session, or recorded at the same point in time: interaction data 130, therapy data 132 and device information 134.

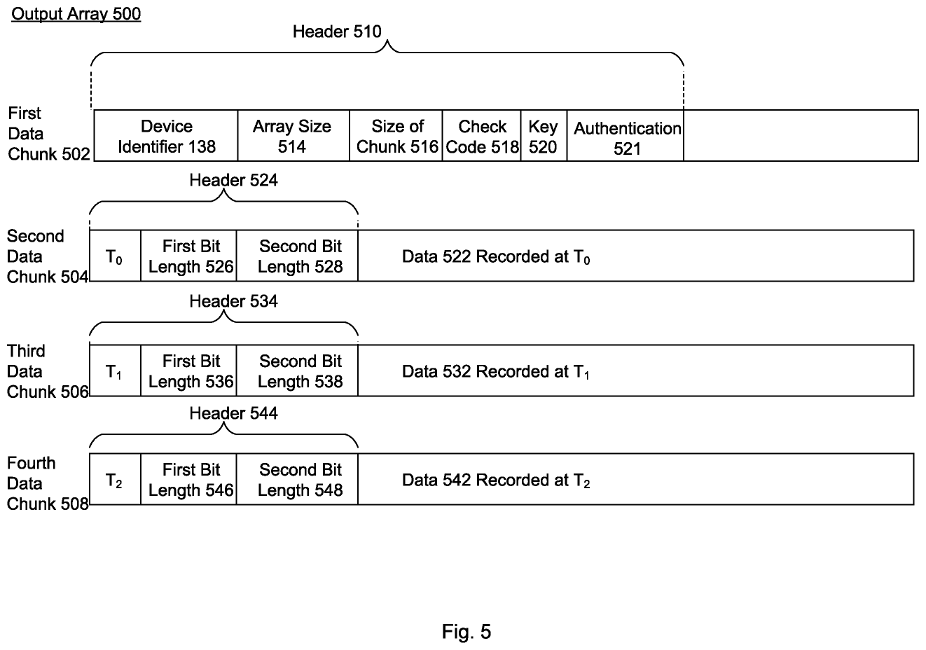

Referring to FIG. 5, the processor(s) 126 in the bundling phase 146 may group multiple arrays generated by the bit packing phase 144, containing multiple time series associated with different timestamps, into a single output array 500 of tuples. Each tuple may include time series metadata. As a result, the processor(s) 126 may send multiple time series to the target system 110 all at the same time, by transmitting the single output array 500 to the target system.

The encoding of the metadata may be described by a serialization format, such as Protocol Buffers, which may include information about the time series such as device settings 136 at the time when therapy data 132 is recorded, time at which the therapy data 132 is recorded, and parameters used during compression, such as the first bit length and the second bit length used in the bit packing phase 144.

Referring to FIG. 5, in one example, the output array 500 may be a large byte array, including one or more small arrays or data chunks 502, 504, 506 and 508. In one example, the output array 500 may represent a QR code of a size of 1024 bytes. In another example, the output array 500 may represent a TCP packet of a size of 65535 bytes.

The processor(s) 126 may insert a header 510 into the first data chunk 502. The header 510 may include overall information that describes the entire output array 500. For example, the header 510 may include one or more of the following: the device identifier 138 that uniquely identifies the data gathering system 100, an array size 514 that describes the total number of data chunks in the output array 500, and a size 516 of each data chunk.

Each time series may be chunked based on the specificity of the transmission medium. Each data chunk may include a header. For example, as shown in FIG. 5, the second data chunk 504 may include data 522 recorded at time T₀, prepended with a header 524. The header 524 may indicate time T₀ at which the data was recorded, and a bit length of each value contained in the data. For example, the header 524 may include a first bit length 526 and a second bit length 528 as used for compressing the data during the bit packing phase. The data 522 may include one or more arrays of data generated by the bit packing phase 144. For instance, the data 522 may include the first output array 406, containing values in the first bit length, and the second output array 408, containing values in the second bit length, generated by the bit packing phase 144, where all values included therein are associated with the same timestamp T₀.

The third data chunk 506 may store data 532 recorded at time T₁, prepended with a header 534 which indicates time T₁ at which the data was recorded, a first bit length 536 and a second bit length 538 as used for compressing the data during the bit packing phase.

Similarly, the fourth data chunk 508 may store data 542 recorded at time T₂, prepended with a header 544 which indicates time T₂ at which the data was recorded, a first bit length 546 and a second bit length 548 as used for compressing the data during the bit packing phase.

The network interface 148, such as the transceiver 149, may transmit the output array 500 in a single transmission, so that all data chunks 502-508 containing data recorded at different points in time may be transmitted simultaneously.

### 2.7.4 Example Compression Process

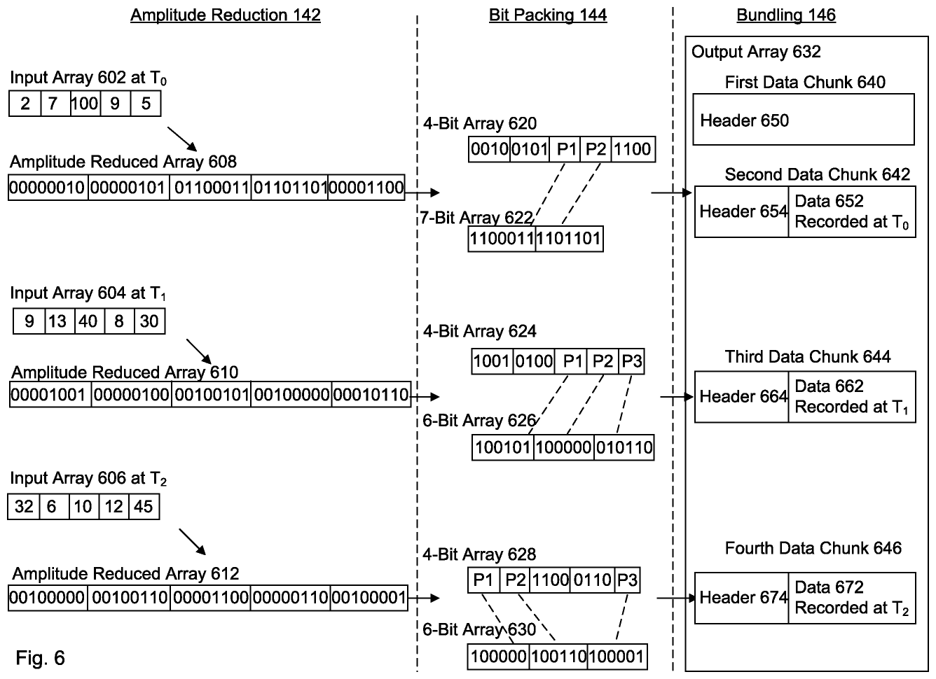

FIG. 6 illustrates an example compression process for processing data recorded during multiple treatment sessions or at different points in time. Data associated with each treatment session or each point in time may be provided in a separate input array. For example, input arrays 602, 604 and 606 may store data recorded at time T₀, T₁ and T₂, respectively. Each time T₀, T₁ and T₂ may represent a single timestamp, a single period of time, or time associated with a single treatment session.

First, in the amplitude reduction phase 142, the processor(s) 126 may perform an XOR operation on every two consecutive values in each input array 602, 604 and 606 to determine their differences, and generate amplitude reduced arrays 608, 610 and 612 to respectively store the differences.

Second, in the bit packing phase 144, with respect to each amplitude reduced array, the processor(s) 126 may determine a first bit length to express each outlier value, and a second bit length to express each standard value. The processor(s) 126 may generate a first array for storing all outlier values, and a second array for storing all standard values. For instance, the processor(s) 126 may process the first amplitude reduced array 608, and generate a 4-bit array 620 to store standard values and a 7-bit array 622 to store outlier values. P1 and P2 in the 4-bit array 620 may refer to addresses in the 7-bit array 622.

Similarly, with respect to the second amplitude reduced array 610, the processor(s) 126 may generate a 4-bit array 624 to store standard values, and a 6-bit array 626 to store outlier values. P1 and P2 in the 4-bit array 624 may refer to addresses in the 6-bit array 626.

Likewise, with respect to the third amplitude reduced array 612, the processor(s) 126 may generate a 4-bit array 628 to store standard values, and a 6-bit array 630 to store outlier values. P1, P2 and P3 in the 4-bit array 628 may refer to addresses in the 6-bit array 630.

Next, in the bundling phase 146, the processor(s) 126 may bundle the above arrays 620, 622, 624, 626, 628 and 630 generated in the bit packing phase 144 into a single output array 632 for simultaneous transmission to the target system 110. The single output array 632 may include a plurality of data chunks 640, 642, 644 and 646. The first data chunk 640 may include a header 650 configured to describe overall information of the output array 632.

Each succeeding data chunk 642, 644 and 646 may store data associated with a single timestamp, a single period of time, or a single treatment session. For example, the second data chunk 642 may store data 652 recorded at T₀, which may include the 4-bit array 620 and 7-bit array 622 generated by the bit packing phase 144, as these arrays 620 and 622 originate from data recorded at T₀. The second data chunk 642 may also include a header 654 which may indicate time T₀ as well as the bit lengths used in the arrays 620 and 622, such as a first bit length of 4 corresponding to the 4-bit array 620, and a second bit length of 7 corresponding to the 7-bit array 622.

Similarly, the third data chunk 644 may store data 662 recorded at T₁, which may include the 4-bit array 624 and 7-bit array 626 generated by the bit packing phase 144, as these arrays 624 and 626 originate from data recorded at T₁. The third data chunk 644 may also include a header 664 indicating time T₁ as well as the bit lengths used in the arrays 624 and 626, such as a first bit length of 4 for expressing values in the 4-bit array 624, and a second bit length of 6 for expressing values in the 6-bit array 626.

Likewise, the fourth data chunk 646 may store data 672 recorded at T₂, which may include the 4-bit array 628 and 6-bit array 630 generated by the bit packing phase 144, as these arrays 628 and 630 originate from data recorded at T₂. The third data chunk 646 may also include a header 674 indicating time T₂ as well as the bit lengths used in the arrays 628 and 630, such as a first bit length of 4 for expressing values in the 4-bit array 628, and a second bit length of 6 for expressing values in the 6-bit array 630.

In one embodiment, the processor(s) 126 may send the output array 632 directly to the network interface 148 for transmission to the target system 110. Alternatively, the processor(s) 126 may execute one or more optional modules 150, described below, before transmitting the output array 632 to the target system 110.

### 2.8 Optional Module

Referring to FIG. 2, the data gathering system 100 may include one or more optional modules 150 configured to perform one or more of the following: integrity check 152, authentication 154 and encryption 156. Anyone or any combination of the optional modules may be selectively performed at the user's discretion either before or after the compression module 140. The user 102 may select any one or more of the optional modules for execution through the user interface 120. The modules typically include programming code for the processor(s) 126 with one or more procedures or functions for generating the desired output based on input to achieve the function of the module.

### 2.8.1 Integrity

To verify integrity of data, the processor(s) 126 may execute check code to perform a checksum verification method or a cyclic redundancy check (CRC) verification method. By doing so, the processor(s) 126 may make sure bits do not get accidentally flipped when stored in the memory.

Further, as shown in FIG. 5, the processor(s) 126 may add the check code 518 into the header 510 of the output array 500 generated by the bundling phase. Once data is transmitted over to the target system 110, the check code 518 may employ the checksum verification method or the CRC verification method to verify integrity of the transmitted data.

### 2.8.2 Authentication

The optional modules 150 may perform one or more of the following authentication 154 related tasks, including, but not limited to, target system authentication, and network authentication, each of which is described below.

### 2.8.2.1 Target System Authentication

In some embodiment, the target system 110, such as the remote server 112, may have a restricted access requiring authentication information in order to gain access, such as to transfer data thereto, to the target system 110. Authentication information may include one or more of the following: a combination of user name and password, and the device identifier 138 of the data gathering system 100, among other possibilities. The device identifier(s) may be unique, enabling the target system 100 to determine an identity of the data gathering system 100. The device identifier(s) may include one or more of the following: a serial or other unique number of the data gathering system 100, and a type or device model of the data gathering system 100, among other possibilities.

The authentication information may be preconfigured and stored in the memory 128. In one embodiment, the authentication information may be provided by the user 102 through keyboard input or audio input. In the event that the authentication information includes the device identifier 138 of the data gathering system 100, the authentication information may be obtained in the manner as described earlier with respect to the device identifier 138.

Referring to FIG. 5, the data gathering system 100 may add the authentication information 521 into the header 510 of the of the output array 500 generated by the bundling phase. Once data is transmitted over to the target system 110, the authentication information may be used to update the target system 110, such as updating user account information stored in the target system 110 based on the transmitted data.

### 2.8.2.2 Network Authentication

To establish a connection with the target system 110 via a network, such as the user's home Wi-Fi, the transceiver 149 may need to provide network authentication information to log into the network. The network authentication information may include, for example, a wireless network name or service set identifier (SSID), and may include a password for the network. The data gathering system 100 may receive the network authentication information from the user through the user interface 120. Alternatively, the data gathering system 100 may receive the network authentication information from the wireless device 114 via a direct Wi-Fi connection, where the wireless device may prompt the user to enter the authentication information of the network, and then transmit the authentication information to the data gathering system 100.

The network authentication information may be stored in the memory 128, and used by the transceiver 149 thereafter for connecting to the network when needed.

### 2.8.3 Encryption

In some implementations, the data gathering system 100 may send encrypted data to a target system 110, so as to have secure communications with the target system 110.

In one example, the data gathering system 100 may have stored an encryption key to encrypt any data before sending it to the target system 110. The processor(s) 126 may encrypt compressed data generated by the compression module by using the encryption key, and instruct the network interface 118 to transmit the encrypted data to the target system 110. Once transmitted, the target system 110 may perform decryption to obtain the data.

As shown in FIG. 5, the processor(s) 126 may add the encryption key 520 to the header 510 of the output array 500 generated by the bundling phase. The encryption key 520 may be used for digital encryption and/or signature.

In another example, the data gathering system 100 may include an encrypting circuit that encrypts the compressed data for sending to the target system 110. The encrypting circuit may be provided in an integrated circuit chip.

### 2.2.2 Flow Diagram

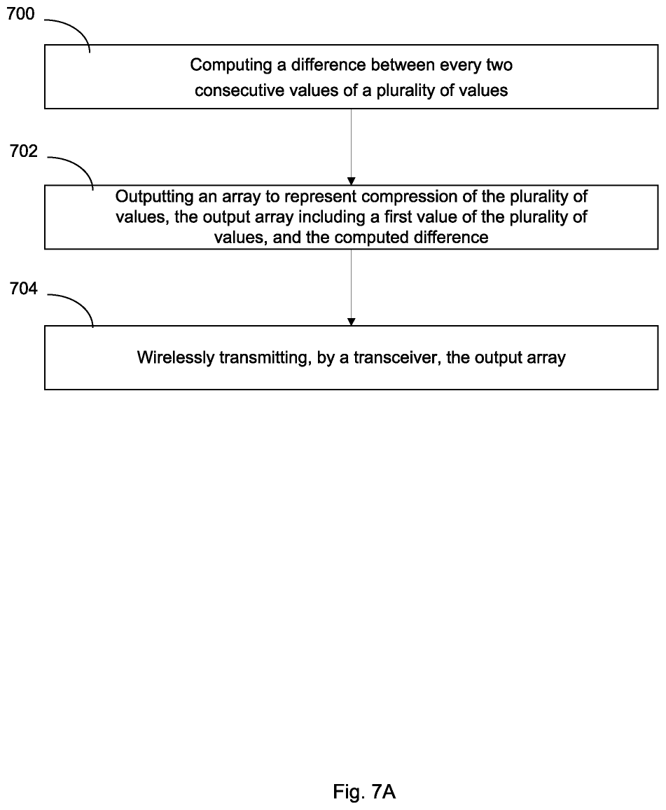

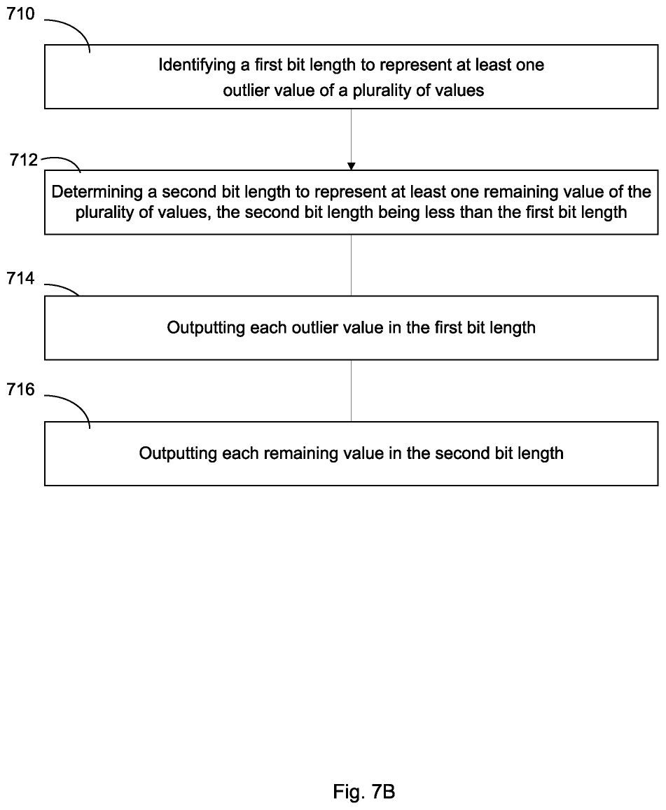

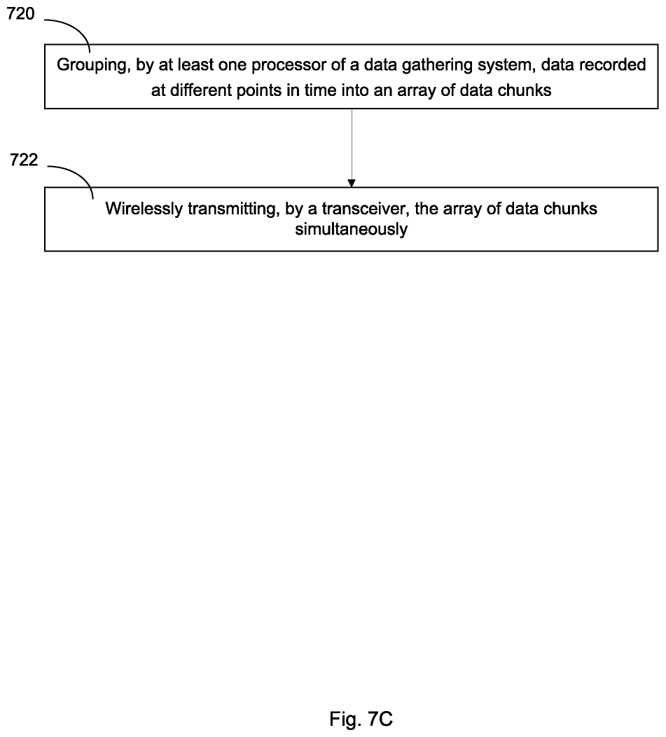

FIG. 7A illustrates a flow diagram of an example process for compressing data in the amplitude reduction phase 142. At 700, the processor(s) 126 may compute a difference between every two consecutive values of a plurality of values. At 702, the processor(s) 126 may output an array to represent compression of the plurality of values. The output array may include a first value of the plurality of values, and the computed difference. At 704, the transceiver 149 may wirelessly transmit the output array.

FIG. 7B illustrates a flow diagram of an example process for compressing data in the bit packing phase 144. This process may reduce a total memory cost for storing the data. At 710, the processor(s) 126 may identify a first bit length to represent at least one outlier value of a plurality of values. The processor(s) 126 may identify the first bit length by determining a minimum number of bits to represent a maximum value in the plurality of values. At 712, the processor(s) 126 may determine a second bit length to represent at least one remaining value of the plurality of values. The second bit length may be less than the first bit length. At 714, the processor(s) 126 may output each outlier value in the first bit length. The processor(s) 126 may identify each outlier value by determining which value out of the plurality of values exceeds the second bit length but fits in the first bit length. At 716, the processor(s) 126 may output each remaining value in the second bit length. The processor(s) 126 may identify each standard value by determining which value out of the plurality of values fits in the second bit length.

FIG. 7C illustrates a flow diagram of an example process for bundling data recorded at different points in time. At 720, the processor(s) 123 may group data recorded at different points in time into an array of data chunks. The array of data chunks may include a first data chunk and a plurality of succeeding data chunks. The first data chunk may include overall information describing the array of data chunks. Each succeeding data chunk may be configured to store the data recorded at one of the different points in time. At 722, the transceiver 149 may wirelessly transmit the array of data chunks simultaneously.

### 3 Technical Advantages

The compression scheme(s) disclosed herein have a wide spectrum of applications, including, but not limited to, a respiratory therapy device with a built-in modem, a respiratory therapy device that stores data on a plug-in dongle which may or may not have a built-in modem, a respiratory therapy device that encodes and displays data that is readable by another device for subsequent wireless transmission, and any device that receives, detects, compresses, or transfers physiological data of a user, examples of which may include a smart watch, a health tracker, a smart phone, a temperature sensor and an ECG/EKG device, among other possibilities.

The present technology disclosed herein presents a technical solution that enables more data transmission with less communication medium (e.g., network) bandwidth.

In particular, the compression scheme disclosed herein reduces the effective payload size of data recorded by the data gathering system, without loss of precision and/or integrity of the underlying data. The reduced payload can be transmitted to the target system over a connection regardless of its bandwidth constraint. Even if the connection has a low bandwidth, the reduced payload can be transmitted intact.

### 4.1 OPTIONAL EXAMPLE TREATMENT SYSTEMS

An example embodiment of the system 100 is discussed in sections 4.1 to 4.5.

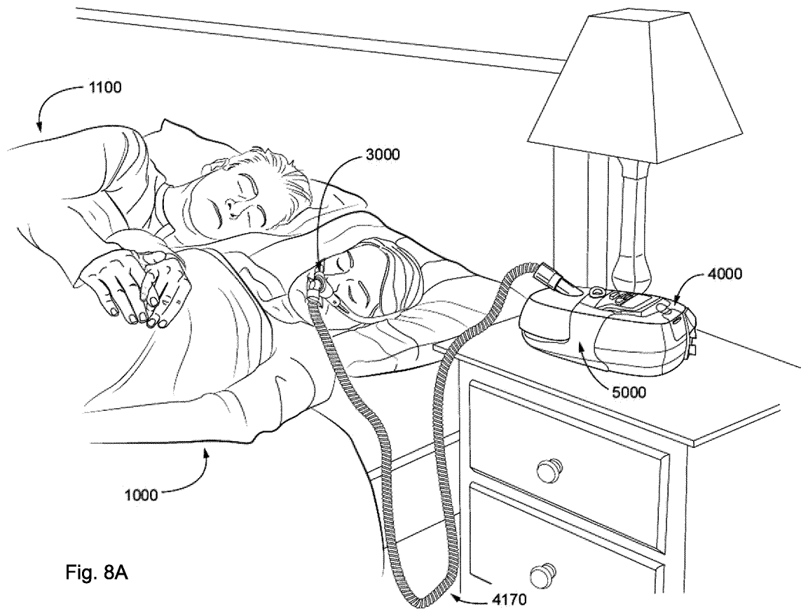

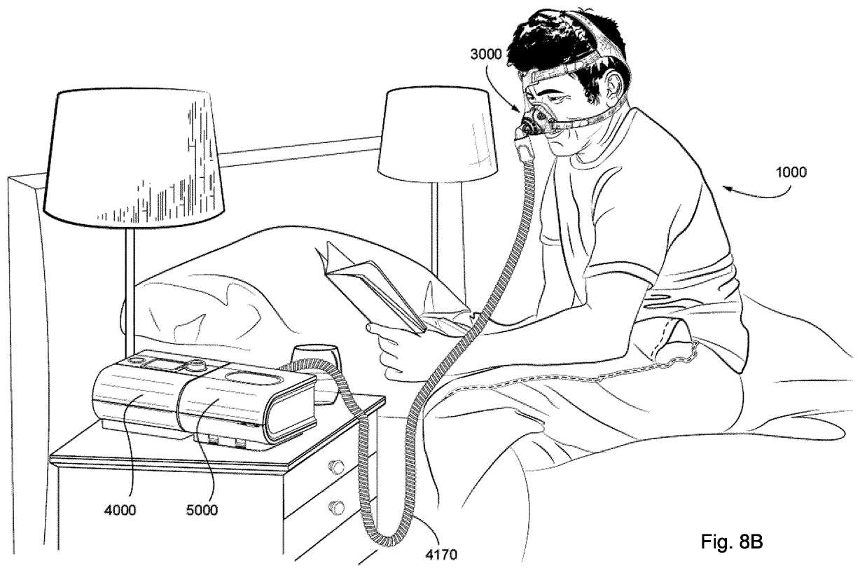

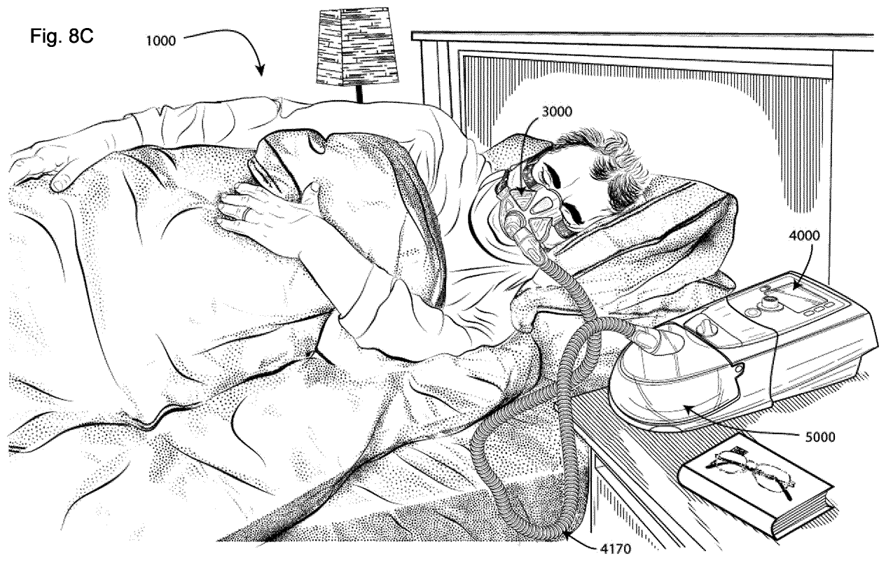

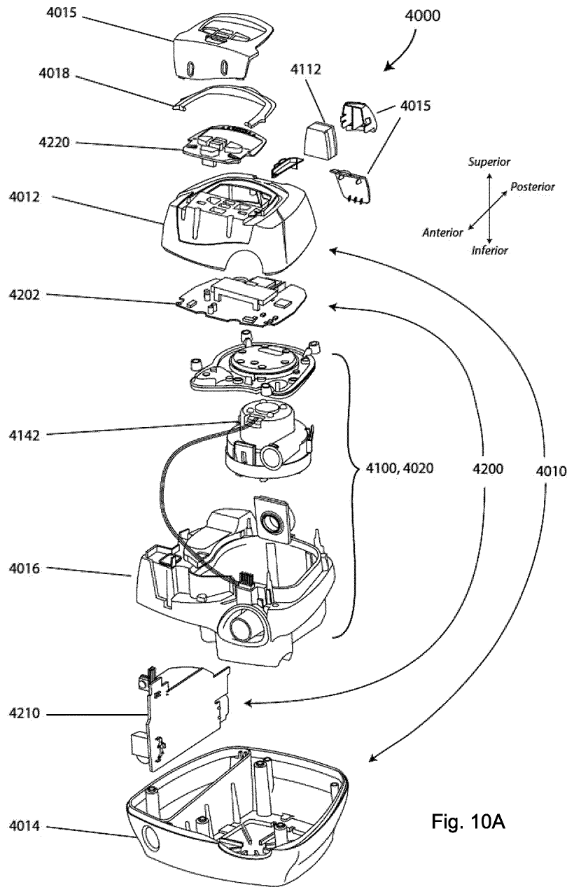

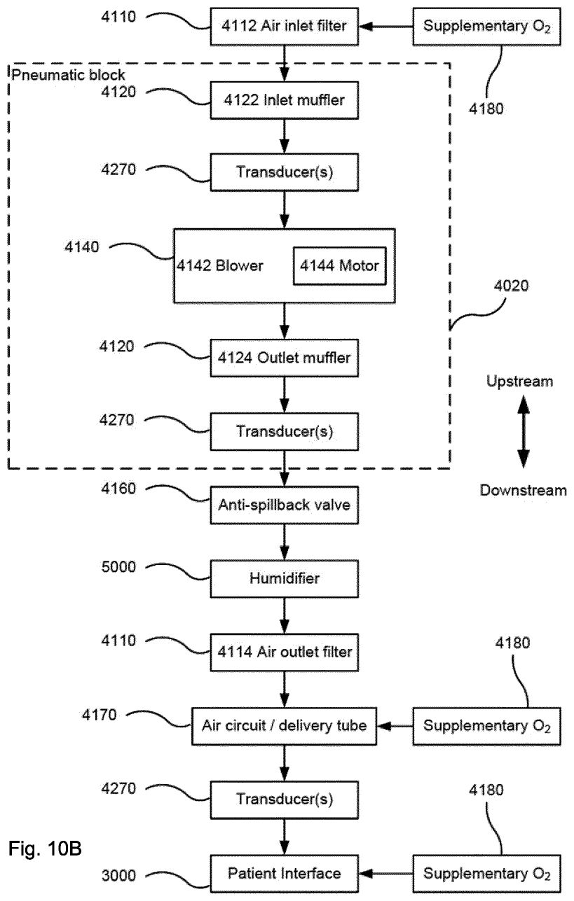

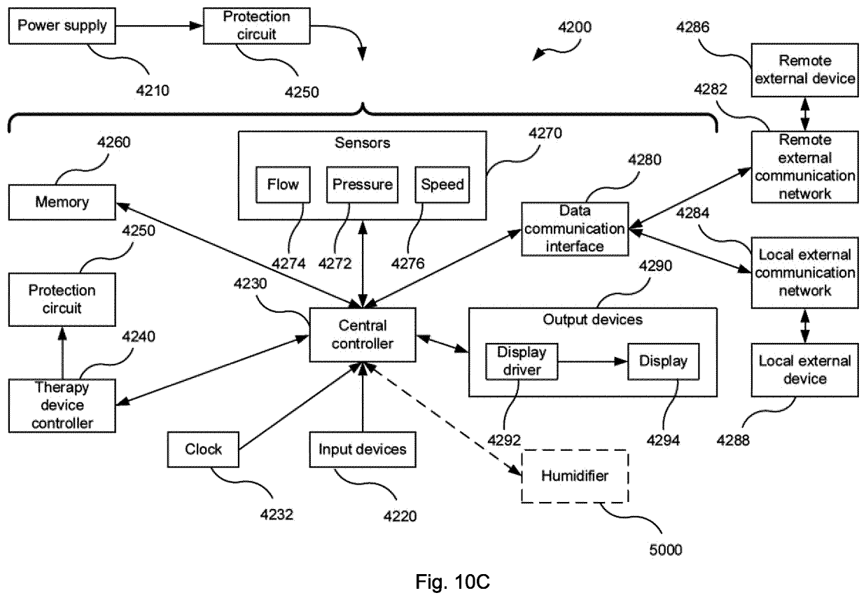

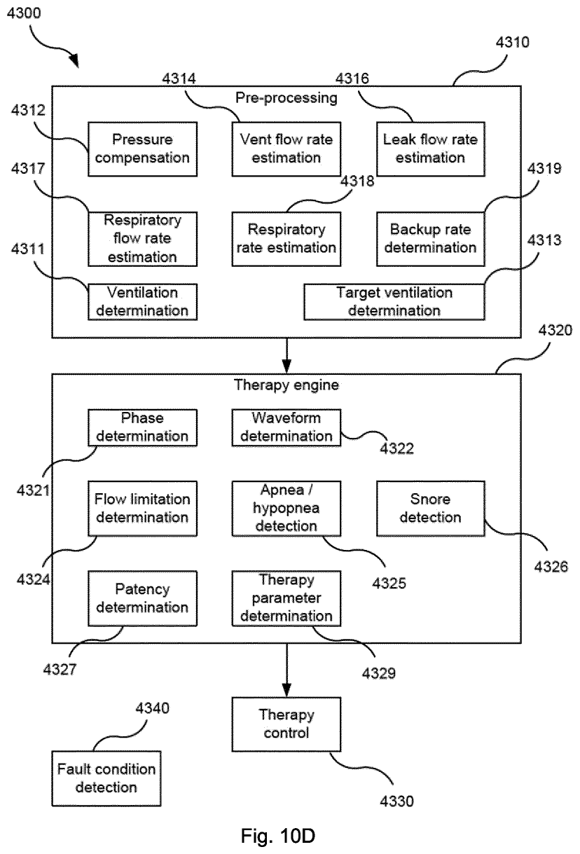

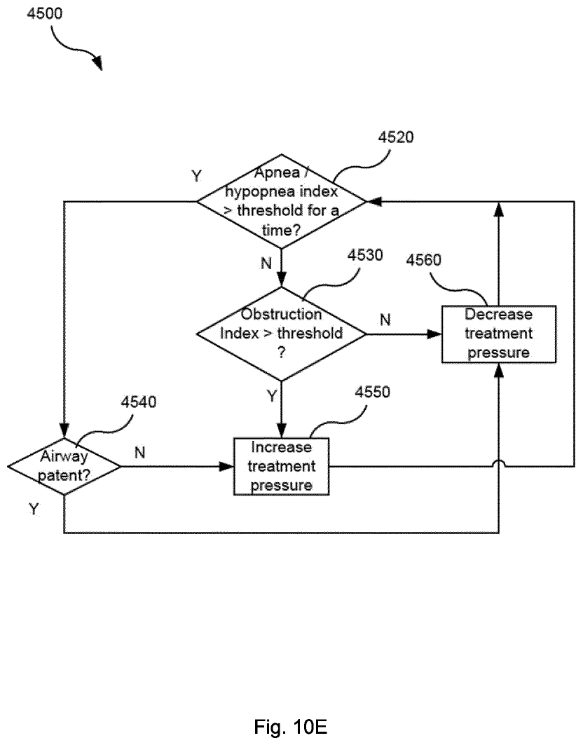

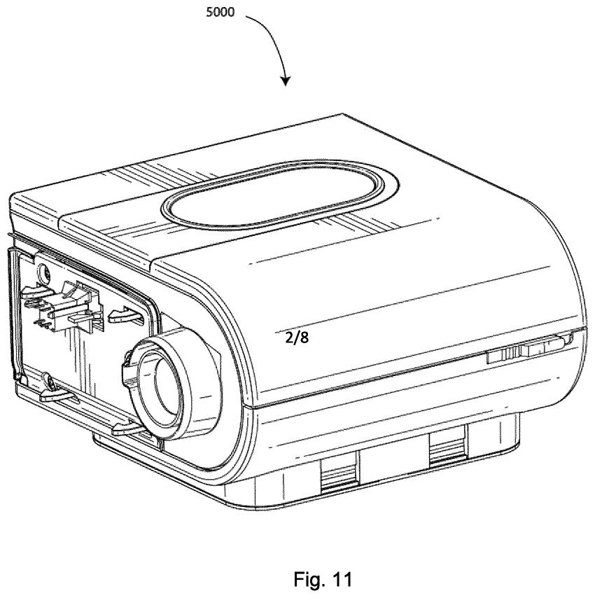

In one form, the system 100 may treat and/or monitor a respiratory disorder. The system 100 may be a respiratory therapy device (RT) such as an RPT device 4000 for supplying a flow of pressurised air to the patient 1000 via an air circuit 4170 leading to a patient interface 3000. The flow of air may be pressure-controlled (for respiratory pressure therapies) or flow-controlled (for flow therapies such as high flow therapy HFT). Thus, RPT devices may also be configured to act as flow therapy devices, such as when using a patient interface that does not use a seal that seals with the patient's respiratory system. In the following description, the RT or RPT device may be considered in reference to FIGS. 8A-11.

### 4.2 PATIENT INTERFACE

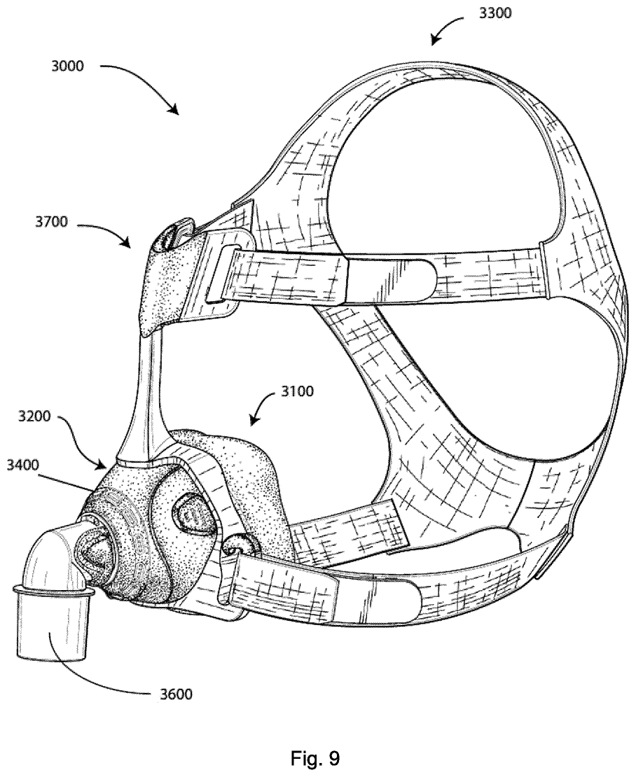

As shown in FIG. 9, a non-invasive patient interface 3000 in accordance with one aspect of the present technology may optionally comprise any of the following functional aspects: a seal-forming structure 3100, a plenum chamber 3200, a positioning and stabilising structure 3300, a vent 3400, a connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to an airway of the patient so as to facilitate the supply of pressurised air to the airway.

### 4.3 RPT DEVICE

An RPT device 4000 in accordance with one aspect of the present technology comprises mechanical and pneumatic components 4100, electrical components 4200 and is programmed to execute one or more algorithms 4300. The RPT device 4000 may have an external housing 4010 formed in two parts, an upper portion 4012 and a lower portion 4014. In one form, the external housing 4010 may include one or more panel(s) 4015. The RPT device 4000 may comprise a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying pressurised air (e.g., a blower 4142), an outlet muffler 4124, and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 4.3.1 RPT Device Mechanical & Pneumatic Components

An RPT device 4000 may comprise one or more of the following components in an integral unit. In an alternative form, one or more of the following components may be located as respective separate units.

### 4.3.1.1 Air Filter(s)

An RPT device 4000 in accordance with one form of the present technology may include an air filter 4110, or a plurality of air filters 4110.

In one form, an air inlet filter 4112 is located at the beginning of the pneumatic path upstream of a pressure generator 4140.

In one form, an air outlet filter 4114, for example an antibacterial filter, is located between an outlet of the pneumatic block 4020 and a patient interface 3000.

### 4.3.1.2 Muffler(s)

An RPT device 4000 in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000.

### 4.3.1.3 Pressure Generator

In one form of the present technology, a pressure generator 4140 for supplying pressurised air is a controllable blower 4142. For example, the blower 4142 may include a brushless DC motor 4144 with one or more impellers housed in a volute. The pressure generator 4140 may be capable of generating a supply or flow of air, for example at about 120 litres/minute, at a positive pressure in a range from about 4 cmH₂O to about 20 cmH₂O, or in other forms up to about 30 cmH₂O.

The pressure generator 4140 is under the control of the therapy device controller 4240.

In other forms, a pressure generator 4140 may be a piston-driven pump, a pressure regulator connected to a high pressure source (e.g., compressed air reservoir), or a bellows.

### 4.3.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 are constructed and arranged to generate data representing respective properties of the air flow, such as a flow rate, a pressure or a temperature, at that point in the pneumatic path.

In one form of the present technology, one or more transducers 4270 are located proximate to the patient interface 3000.

In one form, a signal from a transducer 4270 may be filtered, such as by low-pass, high-pass or band-pass filtering.

### 4.3.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 4.3.1.6 Air Circuit

An air circuit 4170 in accordance with one aspect of the present technology is a conduit or tube constructed and arranged to allow, in use, a flow of air to travel between two components such as the pneumatic block 4020 and the patient interface 3000.

### 4.3.1.7 Oxygen Delivery

In one form of the present technology, supplemental oxygen 4180 is delivered to one or more points in the pneumatic path, such as upstream of the pneumatic block 4020, to the air circuit 4170 and/or to the patient interface 3000.

### 4.3.2 RPT Device Electrical Components

### 4.3.2.1 Power Supply

In one form of the present technology power supply 4210 is internal of the external housing 4010 of the RPT device 4000. In another form of the present technology, power supply 4210 is external of the external housing 4010 of the RPT device 4000.

In one form of the present technology power supply 4210 provides electrical power to the RPT device 4000 only. In another form of the present technology, power supply 4210 provides electrical power to both RPT device 4000 and humidifier 5000.

### 4.3.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form the input device 4220 may be constructed and arranged to allow a person to select a value and/or a menu option.

### 4.3.2.3 Central Controller

In one form of the present technology, the central controller 4230 is a processor suitable to control an RPT device 4000 such as an x86 INTEL processor.

A central controller 4230 suitable to control an RPT device 4000 in accordance with another form of the present technology includes a processor based on ARM Cortex-M processor from ARM Holdings. For example, an STM32 series microcontroller from ST MICROELECTRONICS may be used.

Another central controller 4230 suitable to control an RPT device 4000 in accordance with a further alternative form of the present technology includes a member selected from the family ARM9-based 32-bit RISC CPUs. For example, an STR9 series microcontroller from ST MICROELECTRONICS may be used.

In certain alternative forms of the present technology, a 16-bit RISC CPU may be used as the central controller 4230 for the RPT device 4000. For example, a processor from the MSP430 family of microcontrollers, manufactured by TEXAS INSTRUMENTS, may be used.

In another form of the present technology, the central controller 4230 is a dedicated electronic circuit. In another form, the central controller 4230 is an application-specific integrated circuit (ASIC). In another form, the central controller 4230 comprises discrete electronic components.

The central controller 4230 is configured to receive input signal(s) from one or more transducers 4270, one or more input devices 4220, and the humidifier 5000.

The central controller 4230 is configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300, expressed as computer programs stored in a non-transitory computer readable storage medium, such as memory 4260 or other memory described herein. In some forms of the present technology, as previously discussed, the central controller 4230 may be integrated with an RPT device 4000. However, in some forms of the present technology, some methodologies may be performed by a remotely located device or server such as the server previously mentioned. For example, the remotely located device or server may determine control settings for transfer to a ventilator or other RT device such as by detecting respiratory related events and distinguishing them by type by an analysis of stored data such as from any of the sensors described herein.

While the central controller 4230 may comprise a single controller interacting with various sensors 4270, data communications interface 4280, memory 4260, as well as other devices, the functions of controller 4230 may be distributed among more than one controller. Thus, the term “central” as used herein is not meant to limit the architecture to a single controller or processor that controls the other devices. For example, alternative architectures may include a distributed controller architecture involving more than one controller or processor, which may optionally be directly or indirectly in electronic (wired or wireless) communications with the previously described finger sensor or a server in communication with the finger sensor, such as for implementing any of the methodologies described herein. This may include, for example, a separate local (i.e., within RPT device 4000) or remotely located controller that perform some of the algorithms 4300, or even more than one local or remote memory that stores some of the algorithms. In addition, the algorithms when expressed as computer programs may comprise high level human readable code (e.g., C++, Visual Basic, other object oriented languages, etc.) or low/machine level instructions (Assembler, Verilog, etc.). Depending on the functionality of an algorithm(s), such code or instructions may be burnt in the controller, e.g., an ASIC or DSP, or be a run time executable ported to a DSP or general purpose processor that then becomes specifically programmed to perform the tasks required by the algorithm(s).

### 4.3.2.4 Clock

The RPT device 4000 may include a clock 4232 that is connected to the central controller 4230.

### 4.3.2.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 4.3.2.6 Protection Circuits

An RPT device 4000 in accordance with the present technology may comprise one or more protection circuits 4250.

One form of protection circuit 4250 in accordance with the present technology is an electrical protection circuit.

One form of protection circuit 4250 in accordance with the present technology is a temperature or pressure safety circuit.

### 4.3.2.7 Memory

In accordance with one form of the present technology the RPT device 4000 includes memory 4260, for example non-volatile memory. In some forms, memory 4260 may include battery powered static RAM. In some forms, memory 4260 may include volatile RAM.

Memory 4260 may be located on PCBA 4202. Memory 4260 may be in the form of EEPROM, or NAND flash.

Additionally or alternatively, RPT device 4000 includes a removable form of memory 4260, for example a memory card made in accordance with the Secure Digital (SD) standard.

In one form of the present technology, the memory 4260, such as any of the memories previously described, acts as a non-transitory computer readable storage medium on which is stored computer program instructions expressing the one or more methodologies described herein, such as the one or more algorithms 4300.

### 4.3.2.8 Transducers

Transducers may be internal of the device 4000, or external of the RPT device 4000. External transducers may be located for example on or form part of the air delivery circuit 4170, e.g., at the patient interface 3000. External transducers may be in the form of non-contact sensors such as a Doppler radar movement sensor that transmit or transfer data to the RPT device 4000.

### 4.3.2.8.1 Flow Rate

A flow rate transducer 4274 in accordance with the present technology may be based on a differential pressure transducer, for example, an SDP600 Series differential pressure transducer from SENSIRION. The differential pressure transducer is in fluid communication with the pneumatic circuit, with one of each of the pressure transducers connected to respective first and second points in a flow restricting element.

In one example, a signal representing total flow rate Qt from the flow transducer 4274 is received by the central controller 4230.

### 4.3.2.8.2 Pressure

A pressure transducer 4272 in accordance with the present technology is located in fluid communication with the pneumatic path. An example of a suitable pressure transducer 4272 is a sensor from the HONEYWELL ASDX series. An alternative suitable pressure transducer is a sensor from the NPA Series from GENERAL ELECTRIC.

In use, a signal from the pressure transducer 4272 is received by the central controller 4230. In one form, the signal from the pressure transducer 4272 is filtered prior to being received by the central controller 4230.

### 4.3.2.8.3 Motor Speed

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 4.3.2.9 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g., via Ethernet, or optical fibre) or a wireless protocol (e.g., CDMA, GSM, LTE) to connect to the Internet.

In one form, local external communication network 4284 utilises one or more communication standards, such as Bluetooth, or a consumer infrared protocol and may optionally communicate with any of the sensors described herein.

In one form, remote external device 4286 is one or more computers, for example a cluster of networked computers and/or server as described herein. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 4.3.2.10 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 4.3.2.10.1 Display Driver

A display driver 4292 receives as an input the characters, symbols, or images intended for display on the display 4294, and converts them to commands that cause the display 4294 to display those characters, symbols, or images.

### 4.3.2.10.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 4.3.3 RPT Device Algorithms

### 4.3.3.1 Pre-Processing Module

A pre-processing module 4310 in accordance with the present technology receives, as an input, raw data from a transducer 4270, for example a flow rate sensor 4274 or a pressure sensor 4272, and performs one or more process steps to calculate one or more output values that will be used as an input to another module, for example a therapy engine module 4320.

In one form of the present technology, the output values include the interface or mask pressure Pm, the respiratory flow rate Qr, and the leak flow rate Ql.

In various forms of the present technology, the pre-processing module 4310 comprises one or more of the following algorithms: pressure compensation 4312, vent flow rate estimation 4314, leak flow rate estimation 4316, respiratory flow rate estimation 4317, ventilation determination 4311, target ventilation determination 4313, respiratory rate estimation 4318, and backup rate determination 4319.

### 4.3.3.1.1 Pressure Compensation

In one form of the present technology, a pressure compensation algorithm 4312 receives as an input a signal indicative of the pressure in the pneumatic path proximal to an outlet of the pneumatic block 4020. The pressure compensation algorithm 4312 estimates the pressure drop in the air circuit 4170 and provides as an output an estimated pressure, Pm, in the patient interface 3000.

### 4.3.3.1.2 Vent Flow Rate Estimation

In one form of the present technology, a vent flow rate estimation algorithm 4314 receives as an input an estimated pressure, Pm, in the patient interface 3000 and estimates a vent flow rate of air, Qv, from a vent 3400 in a patient interface 3000.

### 4.3.3.1.3 Leak Flow Rate Estimation

In one form of the present technology, a leak flow rate estimation algorithm 4316 receives as an input a total flow rate Qt and a vent flow rate Qv, and estimates a leak flow rate Ql. In one form, the leak flow rate estimation algorithm 4316 estimates the leak flow rate Ql by calculating an average of the difference between the total flow rate and the vent flow rate Qv over a period sufficiently long to include several breathing cycles, e.g., about 10 seconds.

In one form, the leak flow estimation algorithm 4316 receives as an input a total flow rate Qt, a vent flow rate Qv, and an estimated pressure, Pm, in the patient interface 3000, and estimates a leak flow rate Ql by calculating a leak conductance, and determining a leak flow rate Ql to be a function of leak conductance and the pressure Pm. Leak conductance may be calculated as the quotient of low-pass filtered non-vent flow rate equal to the difference between total flow rate Qt and vent flow rate Qv, and low-pass filtered square root of pressure Pm, where the low-pass filter time constant has a value sufficiently long to include several breathing cycles, e.g., about 10 seconds. The leak flow rate Ql may be estimated as the product of leak conductance and a function of pressure, Pm.

### 4.3.3.1.4 Respiratory Flow Rate Estimation

In one form of the present technology, a respiratory flow rate estimation algorithm 4317 receives as an input a total flow rate, Qt, a vent flow rate, Qv, and a leak flow rate, Ql, and estimates a respiratory flow rate of air, Qr, to the patient, by subtracting the vent flow rate Qv and the leak flow rate Ql from the total flow rate Qt.

In other forms of the present technology, the respiratory flow estimation algorithm 4317 provides a value that acts as a proxy for the respiratory flow rate Qr. Possible proxies for respiratory flow rate include:
Respiratory movement of the chest of the patient 1000
Current drawn by the pressure generator 4140
Motor speed of the pressure generator 4140
Trans-thoracic impedance of the patient 1000

The respiratory flow rate proxy value may be provided by a transducer 4270 in the RPT device 4000, e.g., the motor speed sensor 4276, or a sensor external to the RPT device 4000, such a respiratory movement sensor or a trans-thoracic impedance sensor.

### 4.3.3.1.5 Ventilation Determination

In one form of the present technology, a ventilation determination algorithm 4311 receives an input a respiratory flow rate Qr, and determines a measure Vent indicative of current patient ventilation.

In some implementations, the ventilation determination algorithm 4311 determines a measure of ventilation Vent that is an estimate of actual patient ventilation.

In one such implementation, the measure of ventilation Vent is half the absolute value of respiratory flow, Or, optionally filtered by low-pass filter such as a second order Bessel low-pass filter with a corner frequency of 0.11 Hz.

In one such implementation, the measure of ventilation Vent is an estimate of gross alveolar ventilation (i.e. non-anatomical-deadspace ventilation). This requires an estimate of anatomical deadspace. One can use the patient's height (or arm-span in cases of severe skeletal deformity) as a good predictor of anatomical deadspace. Gross alveolar ventilation is then equal to a measure of actual patient ventilation, e.g., determined as above, less the product of the estimated anatomical deadspace and the estimated spontaneous respiratory rate Rs.

In other implementations, the ventilation determination algorithm 4311 determines a measure of ventilation Vent that is broadly proportional to actual patient ventilation. One such implementation estimates peak respiratory flow rate Qpeak over the inspiratory portion of the cycle. This and many other procedures involving sampling the respiratory flow rate Qr produce measures which are broadly proportional to ventilation, provided the flow rate waveform shape does not vary very much (here, the shape of two breaths is taken to be similar when the flow rate waveforms of the breaths normalised in time and amplitude are similar). Some simple examples include the median positive respiratory flow rate, the median of the absolute value of respiratory flow rate, and the standard deviation of flow rate. Arbitrary linear combinations of arbitrary order statistics of the absolute value of respiratory flow rate using positive coefficients, and even some using both positive and negative coefficients, are approximately proportional to ventilation. Another example is the mean of the respiratory flow rate in the middle K proportion (by time) of the inspiratory portion, where 0<K<1. There is an arbitrarily large number of measures that are exactly proportional to ventilation if the flow rate waveform shape is constant.

In other forms, the ventilation determination algorithm 4311 determines a measure Vent of ventilation that is not based on respiratory flow rate Qr, but is a proxy for the current patient ventilation, such as oxygen saturation (SaO₂), or partial pressure of carbon dioxide (PCO₂), obtained from suitable sensors attached to the patient 1000.

### 4.3.3.1.6 Target Ventilation Determination

In one form of the present technology, a central controller 4230 takes as input the measure of current ventilation, Vent, and executes one or more target ventilation determination algorithms 4313 for the determination of a target value Vtgt for the measure of ventilation.

In some forms of the present technology, there is no target ventilation determination algorithm 4313, and the target ventilation Vtgt is predetermined, for example by hard-coding during configuration of the RPT device 4000 or by manual entry through the input device 4220.

In other forms of the present technology, such as adaptive servo-ventilation (ASV) therapy (described below), the target ventilation determination algorithm 4313 computes the target ventilation Vtgt from a value Vtyp indicative of the typical recent ventilation of the patient 1000.

In some forms of adaptive servo-ventilation therapy, the target ventilation Vtgt is computed as a high proportion of, but less than, the typical recent ventilation Vtyp. The high proportion in such forms may be in the range (80%, 100%), or (85%, 95%), or (87%, 92%).

In other forms of adaptive servo-ventilation therapy, the target ventilation Vtgt is computed as a slightly greater than unity multiple of the typical recent ventilation Vtyp.

The typical recent ventilation Vtyp is the value around which the distribution of the measure of current ventilation Vent over multiple time instants over some predetermined timescale tends to cluster, that is, a measure of the central tendency of the measure of current ventilation over recent history. In one implementation of the target ventilation determination algorithm 4313, the recent history is of the order of several minutes, but in any case should be longer than the timescale of Cheyne-Stokes waxing and waning cycles. The target ventilation determination algorithm 4313 may use any of the variety of well-known measures of central tendency to determine the typical recent ventilation Vtyp from the measure of current ventilation, Vent. One such measure is the output of a low-pass filter on the measure of current ventilation Vent, with time constant equal to one hundred seconds.

### 4.3.3.1.7 Respiratory Rate Estimation

In one form of the present technology, a respiratory rate estimation algorithm 4318 receives as an input a respiratory flow rate, Or, to the patient 1000, and produces an estimate of the spontaneous respiratory rate Rs of the patient.

The respiratory rate estimation algorithm 4318 may estimate the spontaneous respiratory rate Rs over periods when the patient 1000 is breathing spontaneously, i.e., when the RPT device 4000 is not delivering “backup breaths” (described below). In some forms of the present technology, the respiratory rate estimation algorithm 4318 estimates the respiratory rate over periods when servo-assistance (defined as pressure support minus minimum pressure support) is low, in one implementation less than 4 cmH₂O, as such periods are more likely to reflect spontaneous respiratory effort.

In some forms of the present technology, the respiratory rate estimation algorithm 4318 estimates the respiratory rate over periods of asleep breathing, since the respiratory rate during these periods may be substantially different from the respiratory rate during wake. Anxiety typically results in a higher respiratory rate than that prevailing during sleep. When patients focus on their own breathing process, their respiratory rates are typically lower than those during normal wakefulness or during sleep. Techniques such as described in Patent Application no. PCT/AU2010/000894, published as WO 2011/006199, the entire disclosure of which is hereby incorporated herein by reference, may be used to identify periods of awake breathing from the respiratory flow rate, Qr.

In some forms of the present technology, the respiratory rate estimation algorithm 4318 estimates the spontaneous respiratory rate Rs as the reciprocal of one of a variety of well-known statistical measures of central tendency of breath duration Ttot during the period of interest. In such measures it is desirable to reject, or at least be robust to, outliers. One such measure, trimmed mean, in which the lower and upper K proportions of the sorted breath durations are discarded and the mean calculated on the remaining breath durations, is robust to outliers. For example, when K is 0.25, this amounts to discarding the upper and lower quartiles of breath duration Ttot. The median is another robust measure of central tendency, though this can occasionally give unsatisfactory results when the distribution is strongly bimodal. A simple mean may also be employed as a measure of central tendency, though it is sensitive to outliers. An initial interval filtering stage, in which contiguous time intervals corresponding to implausible respiratory rates (e.g., greater than 45 breaths/minute or less than 6 breaths/minute) are excluded as outliers from the mean calculation, may be employed. Other filtering mechanisms which may be used alone or in combination with interval filtering are to exclude any breaths that are not part of a sequence of N successive spontaneous breaths, where N is some small integer (e.g., 3), and to exclude the early and late breaths of a sequence of successive spontaneous breaths, e.g., to exclude the first and last breaths of a sequence of four breaths. The rationale for the latter mechanism is that the first and the last breaths in particular, and the early and late breaths in general, of a sequence of spontaneous breaths may be atypical; for example, the first spontaneous breath may occur as a result of an arousal, and the last spontaneous breath may be longer because of the decreasing respiratory drive which results in the backup breath which ends the sequence of spontaneous breaths.

In some forms of the present technology, the respiratory rate estimation algorithm 4318 makes an initial estimate of the spontaneous respiratory rate Rs using an initial period of estimation, to enable the subsequent processing in the therapy engine module 4320 to begin, and then continuously updates the estimate of the spontaneous respiratory rate Rs using a period of estimation that is longer than the initial period of estimation, to improve statistical robustness. For example, the initial period of estimation may be 20 minutes of suitable spontaneous breaths, but the period of estimation may then progressively increase up to some maximum duration, for example 8 hours. Rather than a rolling window of this duration being used for this estimation, low-pass filters on breath duration may be used, with progressively longer response times (more precisely, progressively lower corner frequencies) as the session proceeds.

In some forms, a suitably processed short-term (e.g., 10-minute) measure of central tendency, such as trimmed mean, may be input to a suitable low-pass filter to give an estimate Rs which changes on the time scale of hours or longer. This has the advantage that potentially large amounts of breath duration data do not need to be stored and processed, as might occur if a trimmed mean needs to be calculated on a moving window of breath duration data lasting hours or days.

In some forms of the present technology, respiratory rates measured over short periods of time, and in particular over one breath, may also be used instead of breath duration in the above-described measures of central tendency, giving generally similar but not identical results.

### 4.3.3.2 Therapy Engine Module

In one form of the present technology, a therapy engine module 4320 receives as inputs one or more of a pressure, Pm, in a patient interface 3000, a respiratory flow rate of air to a patient, Or, and an estimate Rs of the spontaneous respiratory rate, and provides as an output one or more therapy parameters. In various forms, the therapy engine module 4320 comprises one or more of the following algorithms: phase determination 4321, waveform determination 4322, inspiratory flow limitation determination 4324, apnea/hypopnea determination 4325, snore detection 4326, airway patency determination 4327, and therapy parameter determination 4329.

### 4.3.3.2.1 Phase Determination

In one form of the present technology, a phase determination algorithm 4321 receives as an input a signal indicative of respiratory flow, Qr, and provides as an output a phase Φ of a current breathing cycle of a patient 1000.

In some forms, known as discrete phase determination, the phase output Φ is a discrete variable. One implementation of discrete phase determination provides a bi-valued phase output Φ with values of either inhalation or exhalation, for example represented as values of 0 and 0.5 revolutions respectively, upon detecting the start of spontaneous inhalation and exhalation respectively. RPT devices 4000 that “trigger” and “cycle” effectively perform discrete phase determination, since the trigger and cycle points are the instants at which the phase changes from exhalation to inhalation and from inhalation to exhalation, respectively. In one implementation of bi-valued phase determination, the phase output Φ is determined to have a discrete value of 0 (thereby “triggering” the RPT device 4000) when the respiratory flow rate Or has a value that exceeds a positive threshold, and a discrete value of 0.5 revolutions (thereby “cycling” the RPT device 4000) when a respiratory flow rate Qr has a value that is more negative than a negative threshold.

Another implementation of discrete phase determination provides a tri-valued phase output Φ with a value of one of inhalation, mid-inspiratory pause, and exhalation.

In other forms, known as continuous phase determination, the phase output Φ is a continuous value, for example varying from 0 to 1 revolutions, or 0 to 2π radians. RPT devices 4000 that perform continuous phase determination may trigger and cycle when the continuous phase reaches 0 and 0.5 revolutions, respectively. In one implementation of continuous phase determination, a continuous value of phase Φ is determined using a fuzzy logic analysis of the respiratory flow rate Qr. A continuous value of phase determined in this implementation is often referred to as “fuzzy phase”. In one implementation of a fuzzy phase determination algorithm 4321, the following rules are applied to the respiratory flow rate Qr:
1. If the respiratory flow rate is zero and increasing fast then the phase is 0 revolutions.
2. If the respiratory flow rate is large positive and steady then the phase is 0.25 revolutions.
3. If the respiratory flow rate is zero and falling fast, then the phase is 0.5 revolutions.
4. If the respiratory flow rate is large negative and steady then the phase is 0.75 revolutions.
5. If the respiratory flow rate is zero and steady and the 5-second low-pass filtered absolute value of the respiratory flow rate is large then the phase is 0.9 revolutions.
6. If the respiratory flow rate is positive and the phase is expiratory, then the phase is 0 revolutions.
7. If the respiratory flow rate is negative and the phase is inspiratory, then the phase is 0.5 revolutions.
8. If the 5-second low-pass filtered absolute value of the respiratory flow rate is large, the phase is increasing at a steady rate equal to the patient's respiratory rate, low-pass filtered with a time constant of 20 seconds.

The output of each rule may be represented as a vector whose phase is the result of the rule and whose magnitude is the fuzzy extent to which the rule is true. The fuzzy extent to which the respiratory flow rate is “large”, “steady”, etc. is determined with suitable membership functions. The results of the rules, represented as vectors, are then combined by some function such as taking the centroid. In such a combination, the rules may be equally weighted, or differently weighted.

In another implementation of continuous phase determination, the inhalation time Ti and the exhalation time Te are first estimated from the respiratory flow rate Qr. The phase Φ is then determined as the half the proportion of the inhalation time Ti that has elapsed since the previous trigger instant, or 0.5 revolutions plus half the proportion of the exhalation time Te that has elapsed since the previous cycle instant (whichever was more recent).

In some forms of the present technology, suitable for pressure support ventilation therapy (described below), the phase determination algorithm 4321 is configured to trigger even when the respiratory flow rate Or is insignificant, such as during an apnea. As a result, the RPT device 4000 delivers “backup breaths” in the absence of spontaneous respiratory effort from the patient 1000. For such forms, known as spontaneous/timed (S/T) modes, the phase determination algorithm 4321 may make use of the backup rate Rb provided by the backup rate determination algorithm 4319.

A phase determination algorithm 4321 that uses “fuzzy phase” may implement S/T mode using the backup rate Rb by including a “momentum” rule in the fuzzy phase rules. The effect of the momentum rule is to carry the continuous phase forward from exhalation to inhalation at the backup rate Rb if there are no features of respiratory flow rate Qr that would otherwise carry the continuous phase forward through the other rules. In one implementation, the more it is true that the measure of ventilation Vent (described below) is well below a target value Vtgt for ventilation (also described below), the more highly the momentum rule is weighted in the combination. However, as a result of the rapid increase in pressure support in response to mild to moderate hypoventilation (with respect to the target ventilation), the ventilation may be quite close to the target ventilation. It is desirable that the momentum rule is given a low weighting when the ventilation is close to target, to allow the patient to breathe at rates significantly lower than the respiratory rate at other times (when the patient is not in a central apnea) without being unnecessarily pushed to breathe at a higher rate by the ventilator. However, when the momentum rule is given a low weighting when ventilation is above a value which is below but close to the target ventilation, adequate ventilation may easily be achieved at a relatively high pressure support at a rate well below the backup rate. It would be desirable for the backup breaths to be delivered at a higher rate, because this would enable the target ventilation to be delivered at a lower pressure support. This is desirable for a number of reasons, a key one of which is to diminish mask leak.

To summarise, in a fuzzy phase determination algorithm 4321 that implements S/T mode, there is a dilemma in choosing the weighting for the momentum rule incorporating the backup rate Rb: if it is too high, the patient may feel “pushed along” by the backup rate. If it is too low, the pressure support may be excessive. Hence it is desirable to provide methods of implementing S/T mode which do not rely on the momentum rule described above.

A phase determination algorithm 4321 (either discrete, or continuous without a momentum rule) may implement S/T mode using the backup rate Rb in a manner known as timed backup. Timed backup may be implemented as follows: the phase determination algorithm 4321 attempts to detect the start of inhalation due to spontaneous respiratory effort, for example by monitoring the respiratory flow rate Qr as described above. If the start of inhalation due to spontaneous respiratory effort is not detected within a period of time after the last trigger instant whose duration is equal to the reciprocal of the backup rate Rb (an interval known as the backup timing threshold), the phase determination algorithm 4321 sets the phase output Φ to a value of inhalation (thereby triggering the RPT device 4000). Once the RPT device 4000 is triggered, and a backup breath begins to be delivered, the phase determination algorithm 4321 attempts to detect the start of spontaneous exhalation, for example by monitoring the respiratory flow rate Qr, upon which the phase output Φ is set to a value of exhalation (thereby cycling the RPT device 4000).

If the backup rate Rb is increased over time from the SBR to the STBR, as in a variable backup rate system described above, the backup timing threshold starts out longer and gradually becomes shorter. That is, the RPT device 4000 starts out less vigilant and gradually becomes more vigilant to lack of spontaneous respiratory effort as more backup breaths are delivered. Such an RPT device 4000 is less likely to make a patient feel “pushed along” if they would prefer to breathe at a lower than standard rate, while still delivering backup breaths when they are needed.

If the STBR in a variable backup rate system adapts to the patient's estimated spontaneous respiratory rate Rs, as in an adaptive variable backup rate system described above, the backup breaths will be delivered at a rate that adapts to the patient's own recent spontaneous respiratory efforts.

### 4.3.3.2.2 Waveform Determination

In one form of the present technology, the therapy control module 4330 controls a pressure generator 4140 to provide a treatment pressure Pt that varies as a function of phase Φ of a breathing cycle of a patient according to a waveform template Π(Φ).

In one form of the present technology, a waveform determination algorithm 4322 provides a waveform template Π(Φ) with values in the range [0, 1] on the domain of phase values Φ provided by the phase determination algorithm 4321 to be used by the therapy parameter determination algorithm 4329.

In one form, suitable for either discrete or continuously-valued phase, the waveform template Π(Φ) is a square-wave template, having a value of 1 for values of phase up to and including 0.5 revolutions, and a value of 0 for values of phase above 0.5 revolutions. In one form, suitable for continuously-valued phase, the waveform template Π(Φ) comprises two smoothly curved portions, namely a smoothly curved (e.g., raised cosine) rise from 0 to 1 for values of phase up to 0.5 revolutions, and a smoothly curved (e.g., exponential) decay from 1 to 0 for values of phase above 0.5 revolutions. One example of such a “smooth and comfortable” waveform template is the “shark fin” waveform template, in which the rise is a raised cosine, and the smooth decay is quasi-exponential (so that the limit of Π as Φ approaches one revolution is precisely zero).

In some forms of the present technology, the waveform determination algorithm 4322 selects a waveform template Π(Φ) from a library of waveform templates, dependent on a setting of the RPT device 4000. Each waveform template Π(Φ) in the library may be provided as a lookup table of values Π against phase values Φ. In other forms, the waveform determination algorithm 4322 computes a waveform template Π(Φ) “on the fly” using a predetermined functional form, possibly parametrised by one or more parameters (e.g., time constant of an exponentially curved portion). The parameters of the functional form may be predetermined or dependent on a current state of the patient 1000.

In some forms of the present technology, suitable for discrete bi-valued phase of either inhalation (Φ=0 revolutions) or exhalation (Φ=0.5 revolutions), the waveform determination algorithm 4322 computes a waveform template Π “on the fly” as a function of both discrete phase Φ and time t measured since the most recent trigger instant (transition from exhalation to inhalation). In one such form, the waveform determination algorithm 4322 computes the waveform template Π(Φ, t) in two portions (inspiratory and expiratory) as follows:

$$\prod ( Φ , t ) = \{ \begin{matrix}{\prod }_{i} ( t ) , & Φ = 0 \\ {\prod }_{e} ( t - {T}_{i} ) , & Φ = 0.5\end{matrix}$$

where Πᵢ(t) and Πₑ(t) are inspiratory and expiratory portions of the waveform template Π(Φ, t), and Ti is the inhalation time. In one such form, the inspiratory portion Πᵢ(t) of the waveform template is a smooth rise from 0 to 1 parametrised by a rise time, and the expiratory portion Πₑ(t) of the waveform template is a smooth fall from 1 to 0 parametrised by a fall time.

### 4.3.3.2.3 Determination of Inspiratory Flow Limitation

In one form of the present technology, a processor executes one or more algorithms 4324 for the detection of inspiratory flow limitation (partial obstruction).

In one form the algorithm 4324 receives as an input a respiratory flow rate signal Or and provides as an output a metric of the extent to which the inspiratory portion of the breath exhibits inspiratory flow limitation.

In one form of the present technology, the inspiratory portion of each breath is identified based on the phase estimated at each instant. For example, the inspiratory portion of the breath is the values of respiratory flow for which the phase □□ is less than or equal to 0.5. A number of evenly spaced points (for example, sixty-five), representing points in time, are interpolated by an interpolator along the inspiratory flow-time curve for each breath. The curve described by the points is then scaled by a scaler to have unity length (duration/period) and unity area to remove the effects of changing respiratory rate and depth. The scaled breaths are then compared in a comparator with a pre-stored template representing a normal unobstructed breath. Breaths deviating by more than a specified threshold (typically 1 scaled unit) at any time during the inspiration from this template, such as those due to coughs, sighs, swallows and hiccups, as determined by a test element, are rejected. For non-rejected data, a moving average of the first such scaled point is calculated by central controller 4230 for the preceding several inspiratory events. This is repeated over the same inspiratory events for the second such point, and so on. Thus, for example, sixty five scaled data points are generated by central controller 4230, and represent a moving average of the preceding several inspiratory events, e.g., three events. The moving average of continuously updated values of the (e.g., sixty five) points are hereinafter called the “scaled flow”, designated as Qs(t). Alternatively, a single inspiratory event can be utilised rather than a moving average.

From the scaled flow, two shape factors relating to the determination of partial obstruction may be calculated.

Shape factor 1 is the ratio of the mean of the middle (e.g., thirty-two) scaled flow points to the mean overall (e.g., sixty-five) scaled flow points. Where this ratio is in excess of unity, the breath will be taken to be normal. Where the ratio is unity or less, the breath will be taken to be obstructed. A ratio of about 1.17 is taken as a threshold between partially obstructed and unobstructed breathing, and equates to a degree of obstruction that would permit maintenance of adequate oxygenation in a typical user.

Shape factor 2 is calculated as the RMS deviation from unit scaled flow, taken over the middle (e.g., thirty two) points. An RMS deviation of about 0.2 units is taken to be normal. An RMS deviation of zero is taken to be a totally flow-limited breath. The closer the RMS deviation to zero, the breath will be taken to be more flow limited.

Shape factors 1 and 2 may be used as alternatives, or in combination. In other forms of the present technology, the number of sampled points, breaths and middle points may differ from those described above. Furthermore, the threshold values can other than those described.

### 4.3.3.2.4 Determination of Apneas and Hypopneas

In one form of the present technology, a central controller 4230 executes one or more algorithms 4325 for the detection of apneas and/or hypopneas.

In one form, the one or more apnea/hypopnea detection algorithms 4325 receive as an input a respiratory flow rate Or and provide as an output a flag that indicates that an apnea or a hypopnea has been detected.

In one form, an apnea will be said to have been detected when a function of respiratory flow rate Or falls below a flow threshold for a predetermined period of time. The function may determine a peak flow, a relatively short-term mean flow, or a flow intermediate of relatively short-term mean and peak flow, for example an RMS flow. The flow threshold may be a relatively long-term measure of flow.

In one form, a hypopnea will be said to have been detected when a function of respiratory flow rate Qr falls below a second flow threshold for a predetermined period of time. The function may determine a peak flow, a relatively short-term mean flow, or a flow intermediate of relatively short-term mean and peak flow, for example an RMS flow. The second flow threshold may be a relatively long-term measure of flow. The second flow threshold is greater than the flow threshold used to detect apneas.

In one form, such respiratory events may be characterized as central or obstructive based at least in part on the aforementioned finger sensor PPG based type detection.

### 4.3.3.2.5 Detection of Snore

In one form of the present technology, a central controller 4230 executes one or more snore detection algorithms 4326 for the detection of snore.

In one form, the snore detection algorithm 4326 receives as an input a respiratory flow rate signal Qr and provides as an output a metric of the extent to which snoring is present.

The snore detection algorithm 4326 may comprise a step of determining the intensity of the flow rate signal in the range of 30-300 Hz. The snore detection algorithm 4326 may further comprises a step of filtering the respiratory flow rate signal Qr to reduce background noise, e.g., the sound of airflow in the system from the blower 4142.

### 4.3.3.2.6 Determination of Airway Patency

In one form of the present technology, a central controller 4230 executes one or more algorithms 4327 for the determination of airway patency.

In one form, airway patency algorithm 4327 receives as an input a respiratory flow rate signal Or, and determines the power of the signal in the frequency range of about 0.75 Hz and about 3 Hz. The presence of a peak in this frequency range is taken to indicate an open airway. The absence of a peak is taken to be an indication of a closed airway.

In one form, the frequency range within which the peak is sought is the frequency of a small forced oscillation in the treatment pressure Pt. In one implementation, the forced oscillation is of frequency 2 Hz with amplitude about 1 cmH₂O.

In one form, airway patency algorithm 4327 receives as an input a respiratory flow rate signal Or, and determines the presence or absence of a cardiogenic signal. The absence of a cardiogenic signal is taken to be an indication of a closed airway.

### 4.3.3.2.7 Determination of Therapy Parameters

In some forms of the present technology, the central controller 4230 executes one or more therapy parameter determination algorithms 4329 for the determination of one or more therapy parameters using the values returned by one or more of the other algorithms in the therapy engine module 4320.

In one form of the present technology, the therapy parameter is an instantaneous treatment pressure Pt. In one implementation of this form, the therapy parameter determination algorithm 4329 determines the treatment pressure Pt using the equation

$$P t = A \prod ( Φ ) + {P}_{0} \qquad ( 1 )$$

where:
   A is an amplitude,
   [symbol] is the current value of phase;
   [symbol] is the waveform template value (in the range 0 to 1) at the current value of phase, and
   P₀ is a base pressure.

If the waveform determination algorithm 4322 provides the waveform template [symbol] as a lookup table of values indexed by phase [symbol] , the therapy parameter determination algorithm 4329 applies equation (1) by locating the nearest lookup table entry to the current value [symbol] of phase returned by the phase determination algorithm 4321, or by interpolation between the two entries straddling the current value [symbol] of phase.

The values of the amplitude A and the base pressure P₀ may be set by the therapy parameter determination algorithm 4329 depending on the chosen pressure therapy mode in the manner described below.

### 4.3.3.3 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of gas whose mask pressure Pm at the patient interface 3000 is equal to the treatment pressure Pt.

### 4.5 GLOSSARY

For the purposes of the present disclosure, in certain forms of the present technology, one or more of the following definitions may apply. In other forms of the present technology, alternative definitions may apply.

### 4.5.1 General

Air: In certain forms of the present technology, air may be taken to mean atmospheric air, and in other forms of the present technology air may be taken to mean some other combination of breathable gases, e.g., atmospheric air enriched with oxygen.

Respiratory Pressure Therapy (RPT): The delivery of a supply of air to the airways at a treatment pressure that is typically positive with respect to atmosphere.

Continuous Positive Airway Pressure (CPAP) therapy: Respiratory pressure therapy in which the treatment pressure is approximately constant through a breathing cycle of a patient. In some forms, the pressure at the entrance to the airways will be slightly higher during exhalation, and slightly lower during inhalation. In some forms, the pressure will vary between different breathing cycles of the patient, for example, being increased in response to detection of indications of partial upper airway obstruction, and decreased in the absence of indications of partial upper airway obstruction.

Patient: A person, whether or not they are suffering from a respiratory disease.

Automatic Positive Airway Pressure (APAP) therapy: CPAP therapy in which the treatment pressure is automatically adjustable, e.g., from breath to breath, between minimum and maximum limits, depending on the presence or absence of indications of SDB events.

### 4.5.2 Aspects of the Breathing Cycle

Apnea: According to some definitions, an apnea is said to have occurred when respiratory flow rate falls below a predetermined threshold for a duration, e.g., 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort.

Breathing rate, or respiratory rate (Rs): The rate of spontaneous respiration of a patient, usually measured in breaths per minute.

Duty cycle: The ratio of inhalation time, Ti to total breath duration, Ttot.

Effort (breathing): The work done by a spontaneously breathing person attempting to breathe.

Expiratory portion of a breathing cycle: The period from the start of expiratory flow to the start of inspiratory flow.

Flow limitation: The state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Hypopnea: A reduction in flow, but not a cessation of flow. In one form, a hypopnea may be said to have occurred when there is a reduction in flow below a threshold for a duration. In one form in adults, the following either of the following may be regarded as being hypopneas: (i) a 30% reduction in patient breathing for at least 10 seconds plus an associated 4% desaturation; or (ii) a reduction in patient breathing (but less than 50%) for at least 10 seconds, with an associated desaturation of at least 3% or an arousal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed.

Positive End-Expiratory Pressure (PEEP): The pressure above atmosphere in the lungs that exists at the end of expiration.

Peak flow rate (Qpeak): The maximum value of flow during the inspiratory portion of the respiratory flow rate waveform.

Respiratory flow/airflow rate, patient flow/airflow rate (Or): These synonymous terms may be understood to refer to the RPT device's estimate of respiratory airflow rate, as opposed to “true respiratory flow rate” or “true respiratory airflow rate”, which is the actual respiratory flow rate experienced by the patient, usually expressed in litres per minute.

Tidal volume (Vt): The volume of air inhaled or exhaled during normal breathing, when extra effort is not applied.

Inhalation Time (Ti): The duration of the inspiratory portion of the respiratory flow rate waveform.

Exhalation Time (Te): The duration of the expiratory portion of the respiratory flow rate waveform.

(total) Time, or breath duration (Ttot): The total duration between the start of the inspiratory portion of one respiratory flow rate waveform and the start of the inspiratory portion of the following respiratory flow rate waveform.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of the total amount of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 4.5.3 RPT Device Parameters

Flow rate: The instantaneous volume (or mass) of air delivered per unit time. While flow rate and ventilation have the same dimensions of volume or mass per unit time, flow rate is measured over a much shorter period of time. Flow may be nominally positive for the inspiratory portion of a breathing cycle of a patient, and hence negative for the expiratory portion of the breathing cycle of a patient. In some cases, a reference to flow rate will be a reference to a scalar quantity, namely a quantity having magnitude only. In other cases, a reference to flow rate will be a reference to a vector quantity, namely a quantity having both magnitude and direction. Flow rate will be given the symbol Q. ‘Flow rate’ is sometimes shortened to simply ‘flow’. Total flow rate, Qt, is the flow of air leaving the RPT device. Vent flow rate, Qv, is the flow of air leaving a vent to allow washout of exhaled gases. Leak flow rate, Ql, is the flow rate of unintentional leak from a patient interface system. Respiratory flow rate, Or, is the flow of air that is received into the patient's respiratory system.

Leak: The word leak will be taken to be an unintended flow of air. In one example, leak may occur as the result of an incomplete seal between a mask and a patient's face. In another example leak may occur in a swivel elbow to the ambient.

Pressure: Force per unit area. Pressure may be measured in a range of units, including cmH₂O, g-f/cm², hectopascal. 1 cmH₂O is equal to 1 g-f/cm² and is approximately 0.98 hectopascal. In this specification, unless otherwise stated, pressure is given in units of cmH₂O. The pressure in the patient interface (mask pressure) is given the symbol Pm, while the treatment pressure, which represents a target value to be achieved by the mask pressure Pm at the current instant of time, is given the symbol Pt.

### 4.5.4 Terms for Ventilators

Adaptive Servo-Ventilator (ASV): A servo-ventilator that has a changeable rather than a fixed target ventilation. The changeable target ventilation may be learned from some characteristic of the patient, for example, a respiratory characteristic of the patient.

Backup rate: A parameter of a ventilator that establishes the respiratory rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired mask pressure which the ventilator will attempt to achieve at a given time.

End expiratory pressure (EEP): Desired mask pressure which the ventilator will attempt to achieve at the end of the expiratory portion of the breath. If the pressure waveform template Π(Φ) is zero-valued at the end of expiration, i.e., Π(Φ)=0 when Φ=1, the EEP is equal to the EPAP.

IPAP: desired mask pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Servo-assistance: Pressure support minus minimum pressure support.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the inspiratory portion of the breathing cycle by the patient's efforts.

Typical recent ventilation: The typical recent ventilation Vtyp is the value around which recent measures of ventilation over some predetermined timescale tend to cluster, that is, a measure of the central tendency of the measures of ventilation over recent history.

Ventilator: A mechanical device that provides pressure support to a patient to perform some or all of the work of breathing.

### 4.6 OTHER REMARKS

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being preferably used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated by reference to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

Moreover, in interpreting the disclosure, all terms should be interpreted in the broadest reasonable manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular embodiments, it is to be understood that these embodiments are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative embodiments and that other arrangements may be devised without departing from the spirit and scope of the technology.

Although the present invention has been illustrated by reference to specific embodiments, it will be apparent to those skilled in the art that the invention is not limited to the details of the foregoing illustrative embodiments, and that the present technology may be embodied with various changes and modifications without departing from the scope thereof. The present examples are therefore to be considered in all respects as illustrative and not restrictive, the scope of the technology being indicated by the appended claims rather than by the foregoing description, and all changes which come within the meaning and range of equivalency of the claims are therefore intended to be embraced therein. In other words, it is contemplated to cover any and all modifications, variations or equivalents that fall within the scope of the basic underlying principles and whose essential attributes are claimed in this patent application. It will furthermore be understood by the reader of this patent application that the words “comprising” or “comprise” do not exclude other elements or steps, that the words “a” or “an” do not exclude a plurality, and that a single element, such as a computer system, a processor, or another integrated unit may fulfil the functions of several means recited in the claims. Any reference signs in the claims shall not be construed as limiting the respective claims concerned. The terms “first”, “second”, third”, “a”, “b”, “c”, and the like, when used in the description or in the claims are introduced to distinguish between similar elements or steps and are not necessarily describing a sequential or chronological order. Similarly, the terms “top”, “bottom”, “over”, “under”, and the like are introduced for descriptive purposes and not necessarily to denote relative positions. It is to be understood that the terms so used are interchangeable under appropriate circumstances and embodiments of the technology are capable of operating according to the present technology in other sequences, or in orientations different from the one(s) described or illustrated above.

### 4.7 FURTHER EXAMPLES OF THE TECHNOLOGY

The following paragraphs further illustrate example implementations of the present technology described herein.

Example 1. A system, comprising:
a data transfer device for transferring data from the system; and
at least one processor configured to compress a plurality of values for the data transfer device, the at least one processor configured to:
   compute a difference between every two consecutive values of the plurality of values; and
   output an array to represent compression of the plurality of values, the output array including a first value of the plurality of values, and the computed difference.

Example 2. The system of Example 1 wherein the data transfer device comprises any of a wireless transceiver, a removable memory medium, and a display coupled with the at least one processor so as to be configured to display the data in a format readable by a wireless device.

Example 3. The system of Example 2, wherein the format readable by the wireless device includes one or more of a barcode, text and a symbol.

Example 4. The system of Example 3, wherein the barcode includes a quick response (QR) code.

Example 5. The system of any one of Examples 1 to 4, wherein the at least one processor is configured to compute the difference between every two consecutive values by using an XOR function to obtain a differentiating bit sequence.

Example 6. The system of any one of Examples 1 to 5, wherein each value comprises 12 bits.

Example 7. The system of any one of Examples 1 to 6, wherein the plurality of values include the first value and a second value, wherein the output array includes, in a first position, the first value, and wherein the output array includes, in a second position, the computed difference between the first value and the second value.

Example 8. The system of any one of Examples 1 to 7, wherein each element in the output array corresponds to a value of the plurality of values at a corresponding position.

Example 9. The system of Example 8, wherein a first element in a first position of the output array stores the first value of the plurality of values, and wherein a succeeding element in a succeeding position of the output array stores the computed difference between a corresponding value of the plurality of values and its preceding value.

Example 10. The system of any one of Examples 1 to 9, wherein the system includes a respiratory pressure medical device.

Example 11. The system of any one of Examples 1 to 10, wherein the plurality of values include at least one of: interaction data, therapy data, and device information.

Example 12. The system of Example 11, wherein the device information includes one or more of: one or more device settings, one or more device factory settings, a device identifier, and operational data.

Example 13. The system of any one of Examples 1 to 12, wherein the at least one processor is configured to reduce a total memory use for storing the output array, by:
identifying a first bit length to represent at least one outlier value of the output array;
determining a second bit length to represent at least one remaining value of the output array, the second bit length being less than the first bit length;
outputting each outlier value in the first bit length; and
outputting each remaining value in the second bit length.

Example 14. The system of Example 13, wherein the output values are recorded at different points in time, wherein the at least one processor is configured to:
group the output values into an array of data chunks, the array of data chunks including a first data chunk and a plurality of succeeding data chunks, the first data chunk including overall information describing the array of data chunks, each succeeding data chunk being configured to store the output values recorded at one of the different points in time; and
instruct the data transfer device to wirelessly transmit the array of data chunks simultaneously.

Example 15. The system of Example 14, wherein the array of data chunks includes a header, and wherein the header includes at least one of the following: the first bit length and the second bit length.

Example 16. A system, comprising:
a data transfer device for transferring data from the system; and
at least one processor configured to compress a plurality of values for the data transfer device by reducing a total memory use for storing the plurality of values, the at least one processor configured to:
   identify a first bit length to represent at least one outlier value of the plurality of values;
   determine a second bit length to represent at least one remaining value of the plurality of values, the second bit length being less than the first bit length;
   output each outlier value in the first bit length; and
   output each remaining value in the second bit length.

Example 17. The system of Example 16 wherein the data transfer device comprises any of a wireless transceiver, a removable memory medium, and a display coupled with the at least one processor so as to be configured to display the data in a format readable by a wireless device.

Example 18. The system of any one of Examples 16 to 17, wherein each outlier value is a value of the plurality of values that exceeds the second bit length but fits in the first bit length.

Example 19. The system of any one of Examples 16 to 18, wherein each remaining value is a value of the plurality of values that fits in the second bit length.

Example 20. The system of any one of Example 16 to 19,
wherein the total memory cost is a sum of a first product and a second product,
wherein the first product is a product of the first bit length and a total number of the at least one outlier value, and
wherein the second product is a product of the second bit length and a total number of the at least one remaining value.

Example 21. The system of any one of Examples 16 to 20, wherein the at least one processor is configured to:
pad or remove at least one data bit to or from the at least one outlier value of the plurality of values to satisfy the first bit length.

Example 22. The system of any one of Examples 16 to 21, wherein the at least one processor is configured to:
pad or remove at least one data bit to or from the at least one remaining value of the plurality of values to satisfy the second bit length.

Example 23. The system of any one of Examples 16 to 22, wherein the first bit length is based on a bit position of a most significant bit in a maximum value of the plurality of values.

Example 24. A system, comprising:
a transceiver configured to transmit data wirelessly; and
at least one processor configured to:
   group data, recorded at different points in time, into an array of data chunks, the array of data chunks including a first data chunk and a plurality of succeeding data chunks, the first data chunk including overall information describing the array of data chunks, each succeeding data chunk being configured to store the data recorded at one of the different points in time; and
   instruct the transceiver to wirelessly transmit the array of data chunks simultaneously.

Example 25. The system of Example 24, wherein each succeeding data chunk stores at least one of the following: time at which each data is recorded, and a bit length of each value contained in the data.

Example 26. The system of any one of Examples 24 to 25, wherein at least one of the data chunks includes an array of data.

Example 27. The system of any one of Examples 24 to 26, wherein a first data chunk of the array of data chunks includes a header, and wherein the header includes at least one of the following: a device identifier, an array size, a size of each data chunk, a key for encryption and/or signature and a checksum for data integrity.

Example 28. A method comprising:
compressing, by one or more processors of a data gathering system, a plurality of values for data transfer, including:
   computing a difference between every two consecutive values of the plurality of values; and
   outputting an array to represent compression of the plurality of values, the output array including a first value of the plurality of values, and the computed difference; and
controlling a transfer of the output array with a data transfer device configured to transfer data from the data gathering system.

Example 29. The method of Example 28, further comprising reducing a total memory use for storing the output array by:
identifying a first bit length to represent at least one outlier value of the output array;
determining a second bit length to represent at least one remaining value of the output array, the second bit length being less than the first bit length;
outputting each outlier value in the first bit length; and
outputting each remaining value in the second bit length.

Example 30. The method of any one of Examples 28 to 29, wherein the output values are recorded at different points in time, and wherein the method further comprises:
grouping the output values into an array of data chunks, the array of data chunks including a first data chunk and a plurality of succeeding data chunks, the first data chunk including overall information describing the array of data chunks, each succeeding data chunk being configured to store the output values recorded at one of the different points in time; and
instructing the data transfer device to wirelessly transmit the array of data chunks simultaneously.

Example 31. The method of any one of Examples 28 to 30, further comprising:
generating a header for the array of data chunks, and wherein the header includes at least one of the following: the first bit length and the second bit length.

Example 32. A method comprising:
compressing, by one or more processors of a data gathering system, a plurality of values for data transfer to reduce a total memory use for storing the plurality of values, comprising:
   identifying a first bit length to represent at least one outlier value of the plurality of values;
   determining a second bit length to represent at least one remaining value of the plurality of values, the second bit length being less than the first bit length;
   outputting each outlier value in the first bit length; and
   outputting each remaining value in the second bit length; and
controlling transfer of each output outlier value and each output remaining value with a data transfer device configured to transfer data from the data gathering system.

Example 33. The method of Example 32, wherein each outlier value is a value of the plurality of values that exceeds the second bit length but fits in the first bit length.

Example 34. The method of any one of Examples 32 to 33, wherein each remaining value is a value of the plurality of values that fits in the second bit length.

Example 35. The method of any one of Examples 32 to 34,
wherein the total memory cost is a sum of a first product and a second product, wherein the first product is a product of the first bit length and a total number of the at least one outlier value, and
wherein the second product is a product of the second bit length and a total number of the at least one remaining value.

Example 36. The method of any one of Examples 32 to 35, further comprising:
padding or removing at least one data bit to or from the at least one outlier value of the plurality of values to satisfy the first bit length.

Example 37. The method of any one of Examples 32 to 36, further comprising:
padding or removing at least one data bit to or from the at least one remaining value of the plurality of values to satisfy the second bit length.

Example 38. The method of any one of Examples 32 to 37, wherein the first bit length is based on a bit position of a most significant non-zero bit in a maximum value of the plurality of values.

Example 39. A method comprising:
grouping, by one or more processors of a data gathering system, data recorded at different points in time into an array of data chunks, the array of data chunks including a first data chunk and a plurality of succeeding data chunks, the first data chunk including overall information describing the array of data chunks, each succeeding data chunk being configured to store the data recorded at one of the different points in time; and
wirelessly transmitting, by a transceiver, the array of data chunks simultaneously.

Example 40. The method of Example 39, further comprising storing at least one of the following in the array of data chunks: time at which each data is recorded, and a bit length of each value contained in the data.

Example 41. The method of any one of Examples 39 to 40, wherein each data chunk includes an array of data.

Example 42. The method of any one of Examples 39 to 41, wherein a first data chunk of the array of data chunks includes a header, and wherein the header includes at least one of the following: a size of the array, a size of each data chunk, a key for encryption and/or signature, and a checksum for data integrity.

Example 43. A processor-readable medium, having stored thereon processor-executable instructions which, when executed by the one or more processors of the data gathering system, cause the one or more processors to perform the method of any one of Examples 28 to 42.

Example 44. A system, comprising:
a wireless transceiver for transmitting data from the system; and
at least one processor configured to compress a plurality of values for transmission by the wireless transceiver, the at least one processor configured to:
   compute a difference between every two consecutive values of the plurality of values; and
   output an array to represent compression of the plurality of values, the output array including a first value of the plurality of values, and the computed difference.

Example 45. The system of Example 44, wherein the at least one processor is configured to reduce a total memory use for storing the output array, by:
identifying a first bit length to represent at least one outlier value of the output array;
determining a second bit length to represent at least one remaining value of the output array, the second bit length being less than the first bit length;
outputting each outlier value in the first bit length; and
outputting each remaining value in the second bit length.

Example 46. The system of Example 45, wherein the output values are recorded at different points in time, wherein the at least one processor is configured to:
group the output values into an array of data chunks, the array of data chunks including a first data chunk and a plurality of succeeding data chunks, the first data chunk including overall information describing the array of data chunks, each succeeding data chunk being configured to store the output values recorded at one of the different points in time; and
instruct the data transfer device to wirelessly transmit the array of data chunks simultaneously.

Example 47. A system, comprising:
a removable memory medium for receiving data from the system; and
at least one processor configured to compress a plurality of values for storage in the removable memory medium, the at least one processor configured to:
   compute a difference between every two consecutive values of the plurality of values; and
   output an array to represent compression of the plurality of values, the output array including a first value of the plurality of values, and the computed difference.

Example 48. The system of Example 47, wherein the removable memory medium is configured to wirelessly transmit the compressed values.

Example 49. The system of any one of Examples 47 to 48, wherein the at least one processor is configured to reduce a total memory use for storing the output array, by:
identifying a first bit length to represent at least one outlier value of the output array;
determining a second bit length to represent at least one remaining value of the output array, the second bit length being less than the first bit length;
outputting each outlier value in the first bit length; and
outputting each remaining value in the second bit length.

Example 50. The system of Example 49, wherein the output values are recorded at different points in time, wherein the at least one processor is configured to:
group the output values into an array of data chunks, the array of data chunks including a first data chunk and a plurality of succeeding data chunks, the first data chunk including overall information describing the array of data chunks, each succeeding data chunk being configured to store the output values recorded at one of the different points in time; and
instruct the data transfer device to wirelessly transmit the array of data chunks simultaneously.

1. A system, comprising:
a data transfer device for transferring data from the system; and
at least one processor configured to compress a plurality of values for the data transfer device, the at least one processor configured to:
compute a difference between every two consecutive values of the plurality of values; and
output an array to represent compression of the plurality of values, the output array including a first value of the plurality of values, and the computed difference.

2. The system of claim 1 wherein the data transfer device comprises any of a wireless transceiver, a removable memory medium, and a display coupled with the at least one processor so as to be configured to display the data in a format readable by a wireless device.

3. The system of claim 2, wherein the format readable by the wireless device includes one or more of a barcode, text and a symbol.

4. The system of claim 3, wherein the barcode includes a quick response (QR) code.

5. The system of any one of claims 1 to 4, wherein the at least one processor is configured to compute the difference between every two consecutive values by using an XOR function to obtain a differentiating bit sequence.

6. The system of any one of claims 1 to 5, wherein each value comprises 12 bits.

7. The system of any one of claims 1 to 6, wherein the plurality of values includes the first value and a second value, wherein the output array includes, in a first position, the first value, and wherein the output array includes, in a second position, the computed difference between the first value and the second value.

8. The system of any one of claims 1 to 7, wherein each element in the output array corresponds to a value of the plurality of values at a corresponding position.

9. The system of claim 8, wherein a first element in a first position of the output array stores the first value of the plurality of values, and wherein a succeeding element in a succeeding position of the output array stores the computed difference between a corresponding value of the plurality of values and its preceding value.

10. The system of any one of claims 1 to 9, wherein the system includes a respiratory pressure medical device.

11. The system of any one of claims 1 to 10, wherein the plurality of values includes at least one of: interaction data, therapy data, and device information.

12. The system of claim 11, wherein the device information includes one or more of: one or more device settings, one or more device factory settings, a device identifier, and operational data.

13. The system of any one of claims 1 to 12, wherein the at least one processor is configured to reduce a total memory use for storing the output array, by:
identifying a first bit length to represent at least one outlier value of the output array;
determining a second bit length to represent at least one remaining value of the output array, the second bit length being less than the first bit length;
outputting each outlier value in the first bit length; and
outputting each remaining value in the second bit length.

14. The system of claim 13, wherein the output values are recorded at different points in time, wherein the at least one processor is configured to:
group the output values into an array of data chunks, the array of data chunks including a first data chunk and a plurality of succeeding data chunks, the first data chunk including overall information describing the array of data chunks, each succeeding data chunk being configured to store the output values recorded at one of the different points in time; and
instruct the data transfer device to wirelessly transmit the array of data chunks simultaneously.

15. The system of claim 14, wherein the array of data chunks includes a header, and wherein the header includes at least one of the following: the first bit length and the second bit length.

16. A system, comprising:
a data transfer device for transferring data from the system; and
at least one processor configured to compress a plurality of values for the data transfer device by reducing a total memory use for storing the plurality of values, the at least one processor configured to:
identify a first bit length to represent at least one outlier value of the plurality of values;
determine a second bit length to represent at least one remaining value of the plurality of values, the second bit length being less than the first bit length;
output each outlier value in the first bit length; and
output each remaining value in the second bit length.

17. The system of claim 16 wherein the data transfer device comprises any of a wireless transceiver, a removable memory medium, and a display coupled with the at least one processor so as to be configured to display the data in a format readable by a wireless device.

18. The system of any one of claims 16 to 17, wherein each outlier value is a value of the plurality of values that exceeds the second bit length but fits in the first bit length.

19. The system of any one of claims 16 to 18, wherein each remaining value is a value of the plurality of values that fits in the second bit length.

20. The system of any one of claims 16 to 19,
wherein the total memory cost is a sum of a first product and a second product,
wherein the first product is a product of the first bit length and a total number of the at least one outlier value, and
wherein the second product is a product of the second bit length and a total number of the at least one remaining value.

21. The system of any one of claims 16 to 20, wherein the at least one processor is configured to:
pad or remove at least one data bit to or from the at least one outlier value of the plurality of values to satisfy the first bit length.

22. The system of any one of claims 16 to 21, wherein the at least one processor is configured to:
pad or remove at least one data bit to or from the at least one remaining value of the plurality of values to satisfy the second bit length.

23. The system of any one of claims 16 to 22, wherein the first bit length is based on a bit position of a most significant bit in a maximum value of the plurality of values.

24. A system, comprising:
a transceiver configured to transmit data wirelessly; and
at least one processor configured to:
group data, recorded at different points in time, into an array of data chunks, the array of data chunks including a first data chunk and a plurality of succeeding data chunks, the first data chunk including overall information describing the array of data chunks, each succeeding data chunk being configured to store the data recorded at one of the different points in time; and
instruct the transceiver to wirelessly transmit the array of data chunks simultaneously.

25. The system of claim 24, wherein each succeeding data chunk stores at least one of the following:
time at which each data is recorded, and
a bit length of each value contained in the data.

26. The system of any one of claims 24 to 25, wherein at least one of the data chunks includes an array of data.

27. The system of any one of claims 24 to 26, wherein a first data chunk of the array of data chunks includes a header, and wherein the header includes at least one of the following: a device identifier, an array size, a size of each data chunk, a key for encryption and/or signature and a checksum for data integrity.

28. A method comprising:
compressing, by one or more processors of a data gathering system, a plurality of values for data transfer, including:
computing a difference between every two consecutive values of the plurality of values; and
outputting an array to represent compression of the plurality of values, the output array including a first value of the plurality of values, and the computed difference; and
controlling a transfer of the output array with a data transfer device configured to transfer data from the data gathering system.

29. The method of claim 28, further comprising reducing a total memory use for storing the output array by:
identifying a first bit length to represent at least one outlier value of the output array;
determining a second bit length to represent at least one remaining value of the output array, the second bit length being less than the first bit length;
outputting each outlier value in the first bit length; and
outputting each remaining value in the second bit length.

30. The method of any one of claims 28 to 29, wherein the output values are recorded at different points in time, and wherein the method further comprises:
grouping the output values into an array of data chunks, the array of data chunks including a first data chunk and a plurality of succeeding data chunks, the first data chunk including overall information describing the array of data chunks, each succeeding data chunk being configured to store the output values recorded at one of the different points in time; and
instructing the data transfer device to wirelessly transmit the array of data chunks simultaneously.

31. The method of any one of claims 28 to 30, further comprising:
generating a header for the array of data chunks, and wherein the header includes at least one of the following: the first bit length and the second bit length.

32. A method comprising:
compressing, by one or more processors of a data gathering system, a plurality of values for data transfer to reduce a total memory use for storing the plurality of values, comprising:
identifying a first bit length to represent at least one outlier value of the plurality of values;
determining a second bit length to represent at least one remaining value of the plurality of values, the second bit length being less than the first bit length;
outputting each outlier value in the first bit length; and
outputting each remaining value in the second bit length; and
controlling transfer of each output outlier value and each output remaining value with a data transfer device configured to transfer data from the data gathering system.

33. The method of claim 32, wherein each outlier value is a value of the plurality of values that exceeds the second bit length but fits in the first bit length.

34. The method of any one of claims 32 to 33, wherein each remaining value is a value of the plurality of values that fits in the second bit length.

35. The method of any one of claims 32 to 34,
wherein the total memory cost is a sum of a first product and a second product,
wherein the first product is a product of the first bit length and a total number of the at least one outlier value, and
wherein the second product is a product of the second bit length and a total number of the at least one remaining value.

36. The method of any one of claims 32 to 35, further comprising:
padding or removing at least one data bit to or from the at least one outlier value of the plurality of values to satisfy the first bit length.

37. The method of any one of claims 32 to 36, further comprising:
padding or removing at least one data bit to or from the at least one remaining value of the plurality of values to satisfy the second bit length.

38. The method of any one of claims 32 to 37, wherein the first bit length is based on a bit position of a most significant non-zero bit in a maximum value of the plurality of values.

39. A method comprising:
grouping, by one or more processors of a data gathering system, data recorded at different points in time into an array of data chunks, the array of data chunks including a first data chunk and a plurality of succeeding data chunks, the first data chunk including overall information describing the array of data chunks, each succeeding data chunk being configured to store the data recorded at one of the different points in time; and
wirelessly transmitting, by a transceiver, the array of data chunks simultaneously.

40. The method of claim 39, further comprising storing at least one of the following in the array of data chunks:
time at which each data is recorded, and
a bit length of each value contained in the data.

41. The method of any one of claims 39 to 40, wherein each data chunk includes an array of data.

42. The method of any one of claims 39 to 41, wherein a first data chunk of the array of data chunks includes a header, and wherein the header includes at least one of the following: a size of the array, a size of each data chunk, a key for encryption and/or signature, and a checksum for data integrity.

43. A processor-readable medium, having stored thereon processor-executable instructions which, when executed by the one or more processors of the data gathering system, cause the one or more processors to perform the method of any one of claims 28 to 42.

44. A system, comprising:
a wireless transceiver for transmitting data from the system; and
at least one processor configured to compress a plurality of values for transmission by the wireless transceiver, the at least one processor configured to:
compute a difference between every two consecutive values of the plurality of values; and
output an array to represent compression of the plurality of values, the output array including a first value of the plurality of values, and the computed difference.

45. The system of claim 44, wherein the at least one processor is configured to reduce a total memory use for storing the output array, by:
identifying a first bit length to represent at least one outlier value of the output array;
determining a second bit length to represent at least one remaining value of the output array, the second bit length being less than the first bit length;
outputting each outlier value in the first bit length; and
outputting each remaining value in the second bit length.

46. The system of claim 45, wherein the output values are recorded at different points in time, wherein the at least one processor is configured to:
group the output values into an array of data chunks, the array of data chunks including a first data chunk and a plurality of succeeding data chunks, the first data chunk including overall information describing the array of data chunks, each succeeding data chunk being configured to store the output values recorded at one of the different points in time; and
instruct the wireless transceiver to wirelessly transmit the array of data chunks simultaneously.

47. A system, comprising:
a removable memory medium for receiving data from the system; and
at least one processor configured to compress a plurality of values for storage in the removable memory medium, the at least one processor configured to:
compute a difference between every two consecutive values of the plurality of values; and
output an array to represent compression of the plurality of values, the output array including a first value of the plurality of values, and the computed difference.

48. The system of claim 47, wherein the removable memory medium is configured to wirelessly transmit the compressed values.

49. The system of any one of claims 47 to 48, wherein the at least one processor is configured to reduce a total memory use for storing the output array, by:
identifying a first bit length to represent at least one outlier value of the output array;
determining a second bit length to represent at least one remaining value of the output array, the second bit length being less than the first bit length;
outputting each outlier value in the first bit length; and
outputting each remaining value in the second bit length.

50. The system of claim 49, wherein the output values are recorded at different points in time, wherein the at least one processor is configured to:
group the output values into an array of data chunks, the array of data chunks including a first data chunk and a plurality of succeeding data chunks, the first data chunk including overall information describing the array of data chunks, each succeeding data chunk being configured to store the output values recorded at one of the different points in time; and
instruct the wireless transceiver to wirelessly transmit the array of data chunks simultaneously.
